SEQUENCE LISTING (10 sequences)

SEQ ID NO: 1 - DNA, 24 bp, Artificial sequence
  Synthetic
ctcgagagtggacccaactggaag

SEQ ID NO: 2 - DNA, 20 bp, Artificial sequence
  Synthetic
caggctggcagaggtctcag

SEQ ID NO: 3 - DNA, 23 bp, Artificial sequence
  Synthetic
gaacactcttacctgtgcacagc

SEQ ID NO: 4 - DNA, 20 bp, Artificial sequence
  Synthetic
cttgaccgtgaccggcttcc

SEQ ID NO: 5 - DNA, 20 bp, Artificial sequence
  Synthetic
aacgccccactcacctgctg

SEQ ID NO: 6 - DNA, 20 bp, Artificial sequence
  Synthetic
ccttcttggggtcagcacag

SEQ ID NO: 7 - DNA, 20 bp, Artificial sequence
  Synthetic
tgcaccaccaactgcttagc

SEQ ID NO: 8 - DNA, 21 bp, Artificial sequence
  Synthetic
ggcatggactgtggtcatgag

SEQ ID NO: 9 - DNA, 24 bp, Artificial sequence
  Synthetic
cctaatagttggggcttatatgac

SEQ ID NO: 10 - DNA, 20 bp, Artificial sequence
  Synthetic
gcctaaaaccacagtgggag

(19) United States
(12) Patent Application Publication
(10) Pub. No.: US 2022/0031820 A1
(43) Pub. Date: Feb. 3, 2022

(54) MODULATION OF GRANZYME K ACTIVITY IN THE TREATMENT OF SKIN CONDITIONS

(71) Applicant: THE UNIVERSITY OF BRITISH COLUMBIA, Vancouver, British Columbia (CA)

(72) Inventors: David J. GRANVILLE, Vancouver, British Columbia (CA); Christopher TURNER, Vancouver, British Columbia (CA); Matthew ZEGLINSKI, Vancouver, British Columbia (CA); Katlyn RICHARDSON, Vancouver, British Columbia (CA); Sho HIROYASU, Vancouver, British Columbia (CA)

(73) Assignee: THE UNIVERSITY OF BRITISH COLUMBIA, Vancouver, British Columbia (CA)

(21) Appl. No.: 17/279,442

(22) PCT Filed: Sep. 24, 2019
(86) PCT No.: PCT/CA2019/051359

§ 371 Date: Mar. 24, 2021

Related U.S. Application Data
(60) Provisional application No. 62/735,414, filed on Sep. 24, 2018.
(60) Provisional application No. 62/851,790, filed on May 23, 2019.

(51) Int. Cl. A61K 38/57

(52) U.S. Cl. CPC A61K 38/57

(57) ABSTRACT

Methods for treating inflammatory skin conditions, such as atopic dermatitis and psoriasis, and for promoting skin wound healing and for treating skin wounds, such as thermal and pressure injury, by reducing the activity of Granzyme K.

### CROSS-REFERENCE TO RELATED APPLICATIONS

This application claims priority to U.S. Application No. 62/735,414, filed Sep. 24, 2018, and U.S. Application No. 62/851,790, filed May 23, 2019, each application expressly incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 70381_Seq_Final_2019-09-20.txt. The text file is 4,096 KB; was created on 2019-09-20 and is being submitted via EFS-Web with the filing of the specification.

### BACKGROUND OF THE INVENTION

Granule-secreted enzymes (granzymes) are a family of serine proteases long proposed to contribute to perforin-dependent cytotoxic T lymphocyte (CTL) and natural killer (NK) granule exocytosis-mediated cell death (Lobe et al., 1986, Masson and Tschopp, 1987, Tschopp et al., 1986). There are five granzymes in humans: Granzyme A (tryptase), Granzyme B (aspartase), Granzyme H (chymase), Granzyme K (GzmK; tryptase) and Granzyme M (metase). Each granzyme is uniquely expressed by different cell types, and each possesses separate substrate specificities and function(s) (Reviewed in (Turner et al., 2017a, Voskoboinik et al., 2015)).

Emerging evidence challenges the notion that GzmK is cytotoxic and suggests it may actually act to promote pro-inflammatory cytokine release (Joeckel et al., 2017, Joeckel et al., 2011). Although GzmK occurs at low levels in the plasma of healthy individuals, it is acutely elevated in response to viral infection (Bade et al., 2005), allergic asthma, pneumonia (Bratke et al., 2008), sepsis (Rucevic et al., 2007) and endotoxemia (Wensink et al., 2016). Mice infected with either lymphocytic choriomeningitis (Joeckel et al., 2017) or Chikungunya virus (Wilson et al., 2017) also show increased GzmK expression in plasma derived CTLs and plasma respectively. GzmK−/− mice exhibit reduced foot swelling in response to Chikungunya virus infection (Wilson et al., 2017). Exposure of cultured lung fibroblasts and endothelial cells to GzmK stimulates pro-inflammatory cytokine release that is dependent on PAR-1 activation (Cooper et al., 2011, Sharma et al., 2016). GzmK also induces IL-1 production in macrophages (Joeckel et al., 2011).

Inflammation plays a key role in the development of excessive scarring and painful skin contractures caused by thermal/burn injury. Burn healing requires an intricate coordination of events involving interaction between multiple cell types and the extracellular microenvironment. Curbing excessive inflammation is a major strategy to reduce secondary burn wound expansion, scarring and fibrosis. By augmenting inflammation, GzmK may provide an important contribution to the healing of burn wounds. Aberrant immune cell infiltration and activity also plays a key role in the onset and/or progression of other skin conditions including psoriasis, dermatitis and other forms of wound healing.

A need exists for effective methods for treating inflammatory skin conditions, for treating skins wounds, and for promoting skin wound healing. The present invention fulfills this need and provides further related advantages.

### SUMMARY OF THE INVENTION

The present invention provides methods for treating inflammatory skin conditions, treating skin wounds, and promoting skin wound healing by reducing the activity of Granzyme K (GzmK).

In one aspect, the invention provides a method of treating an inflammatory skin condition in a subject, comprising reducing the activity of Granzyme K in a subject, thereby treating the inflammatory skin condition.

In another aspect, the invention provides a method of treating a wound in a subject, comprising reducing the activity of Granzyme K in a subject, thereby treating the wound.

In certain embodiments of the above methods, reducing the activity of Granzyme K comprises administering an effective amount of a Granzyme K inhibitor to the subject.

In related embodiments, the invention provides a method of treating an inflammatory skin condition in a subject, comprising administering an effective amount of a Granzyme K inhibitor to the subject, thereby treating the inflammatory skin condition, and a method of treating a wound in a subject, comprising administering an effective amount of a Granzyme K inhibitor to the subject, thereby treating the wound.

In a further aspect, the invention provides methods for promoting wound healing. In one embodiment, the invention provides a method of promoting wound healing in a subject, comprising inhibiting cleavage of syndecan-1 in keratinocytes by reducing the activity of Granzyme K in the subject. In another embodiment, the invention provides a method of promoting wound healing in a subject, comprising reducing pro-inflammatory cytokine response in keratinocytes, fibroblasts, macrophages, and/or endothelial cells by reducing the activity of Granzyme K in the subject. In further embodiment, the invention provides a method of promoting wound healing in a subject, comprising inhibiting cleavage of syndecan-1 by administering an effective amount of Granzyme K inhibitor to the subject. In yet a further embodiment, the invention provides a method of promoting wound healing in a subject, comprising reducing pro-inflammatory cytokine response in keratinocytes, fibroblasts, macrophages, and/or endothelial cells by administering an effective amount of Granzyme K inhibitor to the subject.

In another aspect, the invention provides methods for promoting re-epithelization. In one embodiment, the invention provides a method for promoting wound re-epithelization, comprising reducing the activity of Granzyme K in keratinocytes proximate to the wound. In another embodiment, the invention provides a method for promoting wound re-epithelization in a subject, comprising inhibiting cleavage of syndecan-1 in a keratinocyte by administering an effective amount of Granzyme K inhibitor to the subject. In a further embodiment, the invention provides a method for promoting wound re-epithelization in a subject, comprising administering an effective amount of Granzyme K inhibitor to the subject. In yet another embodiment, the invention provides a method of stimulating re-epithelialization, comprising inhibiting syndecan-1 cleavage in the keratinocyte by reducing the activity of GzmK in the wounded or damaged tissue area.

In a further aspect, the invention provides a method of preventing vascular permeability (leakage) in a subject, comprising Granzyme K-mediated immune cell recruitment and endothelial pro-inflammatory response in vessels located at the site of injury, by reducing the activity of Granzyme K.

In another aspect, the invention provides a method of converting a pro-inflammatory phenotype to a pro-healing wound repair phenotype, comprising reducing pro-inflammatory cytokine responses in keratinocytes, fibroblasts, macrophages, and/or endothelial cells by reducing the activity of Granzyme K in the wounded or damaged tissue area.

Inflammatory skin conditions treatable by the above methods include psoriasis and atopic dermatitis.

Wounds treatable by the above methods include burn wounds, chronic wounds, acute wounds, pressure injury wounds, and ischemic injury wounds.

In the above methods, suitable Granzyme K inhibitors includes small molecules, nucleic acid molecules, peptides, and antibodies. Representative Granzyme K inhibitors include inter-alpha inhibitor protein (IαIp) and bikunin. In the methods, the inhibitors can be administered topically or systemically.

In yet another aspect, the invention provides methods for screening compounds for their ability to treat an inflammatory skin condition or to promote wound healing. In one embodiment, the invention provides a method for screening a candidate compound for its ability to treat an inflammatory skin condition or to promote wound healing, comprising contacting the candidate compound with Granzyme K in vitro, wherein inhibition of Granzyme K activity compared to Granzyme K that has not been contacted with the candidate compound indicates that the candidate compound is a compound that may be useful for the treatment of the inflammatory skin condition or wound. In certain embodiments, the candidate compound selectively inhibits Granzyme K and does not substantially inhibit Granzyme A at the same compound concentration.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIGS. 1A-1F illustrate that GzmK is elevated in human burn tissue. FIG. 1A shows GzmK immunohistochemistry and compares healthy skin and burn with negative control. GzmK+ cells associated with inflammatory cell infiltrate (arrowhead) and dermal-epidermal junction (200 μm size bars). FIG. 1B shows that GzmK co-localized with CD68 in human burn inflammatory cell infiltrate (brightness indicates GzmK/CD68 co-localization. FIGS. 1A and 1B images from patient 2 (d21 post-injury) (50 μm size bars). FIG. 1C compares GzmK immune-fluorescence in THP-1 cells polarized to M0, then classically (M1) or alternatively (M2a) activated (10 μm size bars). FIG. 1D shows RT-PCR of macrophage mRNA. FIG. 1E shows ELISA GzmK detection in culture supernatants 24 hour post-incubation in serum-free medium (ng GzmK per mg total cell protein) (Mean±SD, n=3 per group). FIG. 1F shows GzmK immunohistochemistry in mice burns (WT and GzmK−/− mice at d3 and d6, with control) (20 μm size bars) (1F). Negative control in FIGS. 1A, 1B and 1F are secondary antibodies only.

FIGS. 2A-2F illustrates that GzmK−/− mice showed improved wound healing. Photographic comparison of thermal injuries in GzmK−/− and WT mice over time (D1, d3, d6, d9, and d12) (5 mm size bars) (2A). Quantitative analysis of macroscopic wound area (GzmK−/− (dashed line) and WT (solid line) mice; data presented as mean±SEM (n≥6 mice per group)) (2B). Quantitative analysis of wound gape (mm) as measured from hematoxylin and eosin (H&E) stained slides. Data presented as mean±SEM (n=5 per group) (2C). Representative H&E images of wounds d6 post-injury for GzmK−/− and WT are compared (200 μm size bars) (2D). iNOS (M1 macrophage) staining of wounds d3 and d6 post-injury for GzmK−/− and WT mice are compared (200 μm size bars) (2E). M1 macrophage quantification d3 and d6 post-injury for GzmK−/− and WT mice (data presented as mean±SEM (n=5 per group)). *P<0.05, **P<0.005, compared to WT controls and calculated by Student's t-test (2F).

FIGS. 3A-3F illustrate that GzmK−/− mice show improved re-epithelialization and tissue repair. Re-epithelialization of burns at d3 and d6 post-injury for GzmK−/− and WT mice (3A). Scabs falling off provides an indirect re-epithelialization measure (GzmK−/− (dashed line; n=18) and WT mice (solid line; n=15) (3B). Representative Masson's trichrome, Collagen I and Collagen III staining in mouse burn granulation tissue for GzmK−/− and WT mice (d14 post-injury; n=6) (100 um size bar); Masson's trichrome quantification (3D) and Collagen VIII ratio (3E) for GzmK−/− and WT mice (d14 post-injury; n=6) (100 um size bar). Minimum wound breaking force at d21 and d41 post-injury for GzmK−/− and WT mice, n≥6 mice per group (3F). Data in 3A, 3D, and 3E presented as mean±SEM (n≥5 mice per group). Mean plus each individual data point (n≥5 mice per group). *P<0.05, **P<0.005, compared to WT and calculated by Student's t-test (3F).

FIGS. 4A-4G illustrate that GzmK impairs keratinocyte wound healing in vitro and induces pro-inflammatory cytokine expression. In an ECIS wound assay, cells incubated with 10 nM (300 ng/mL), 25 nM (600 ng/mL) rhGzmK or untreated, n=3, performed three times (compares resistance over time for HaCaT and skin fibroblast) (4A). IL-6 ELISA of cell supernatants (compares IL-6 (pg/10⁵ cells) for HaCaT and skin fibroblast) (4B-4E). IL-1β ELISA of macrophage supernatants (compares IL-1β (μg/mg) for macrophage and M1, respectively) (4F and 4G). Cells in FIGS. 4C, 4E, 4F, and 4G incubated with 50 nM rhGzmK. PAR-1 inhibitor ATAP-2 at 5 μg/mL; GzmK inhibitor IαIp at 4 μM; Heat inactivated GzmK=hiGzmK (presented as mean±SD (n=3), performed twice); IL-6 data presented as pg per 10⁵ cells, and IL-1β as pg per mg cell protein. *P<0.05, **P<0.005, compared to untreated; statistics by Student's t-test, except (4A) 2 way ANOVA.

FIGS. 5A-5C illustrate altered inflammatory cell infiltration and cytokine expression in murine burn wounds. ELISA detection of pro-inflammatory cytokines in mice burn tissue (compares IL-6 and IL-1β for WT and GzmK−/− mice at d3 and d6) (data presented as pg/mg cell protein (n≥3 per group) (5A). Gene expression in mice burn tissue at d3 and d6 post-injury for MCP-1, ICAM-1, and VCAM-1 (presented as fold increase over WT samples (n≥3 per group)) (5B). FIG. 5C compares inflammatory cell infiltrate, F4/80 (macrophages), CD3 (T-cells) and NCR1 (NK cells) positive cells in mouse burn tissue at d3 and d6 post-injury (size bars at 100 μm H&E) and 15 μm (IHC); data presented as percentage of cells relative to WT unwounded skin controls, showing mean±SD (n=6). *P<0.05, **P<0.005, compared to WT; statistics by 2 way ANOVA (5C).

FIG. 6 is a schematic illustration of the mechanism of GzmK in impaired thermal injury repair. (1) Following thermal injury in skin, monocytes and resident dermal macrophages are recruited to the site of injury and classically activated. GzmK expression is up-regulated in M1, with some secreted into the wound area (2). GzmK inhibits re-epithelialization (3) and induces pro-inflammatory cytokine release from M1 macrophages, keratinocytes, skin fibroblasts and endothelial cells (4). Endothelial cells also secrete chemokines and adhesion molecules in response to GzmK exposure (5), leading for an up-regulation of monocyte recruitment to the wound (6). Together, GzmK induces an enhanced burn induced pro-inflammatory response, contributing to a delay in wound healing.

FIG. 7 is a macroscopic analysis showing reduced wound gape in GzmK−/− mice compared to WT mice. GzmK−/− (dashed line) and WT (solid line) mice. * P<0.05, ** P<0.005, n≥10 mice per group.

FIGS. 8A-8C show that GzmK is non-cytotoxic to keratinocytes, skin fibroblasts, and macrophages, respectively. Trypan blue dye exclusion was quantified as a measure of cell viability. Data presented as percentage of viable cells per treatment group (n≥3 per group).

FIGS. 9A and 9B compare GzmK immunohistochemistry in human lesional atopic dermatitis tissue showing GzmK+ cells elevated in lesional atopic dermatitis tissue (9B) compared to healthy skin controls (9A).

FIGS. 10A and 10B compare GzmK immunohistochemistry and (10B) TBO (mast cell) (10A) sequential staining of human atopic dermatitis tissue showing that a majority of mast cells express GzmK, but that other cell types also express GzmK.

FIG. 11 illustrates the OXA-induced dermatitis mouse model oxazolone exposure schedule described and used herein.

FIG. 12 illustrates a scaling assessment in the ears of OXA-induced dermatitis mice comparing WT mice and GzmK−/− mice, which display reduced scaling compared WT mice.

FIG. 13 illustrates an erosion assessment in the ears of OXA-induced dermatitis mice comparing WT mice and GzmK−/− mice showing that erosion is initially worse in GzmK−/− mice, but is significantly reduced from d17 compared to WT controls.

FIG. 14 illustrates an erythema assessment in the ears of OXA-induced dermatitis mice comparing WT mice and GzmK−/− mice showing that erythema is initially worse in GzmK−/− mice, but is reduced from d17 compared to WT controls.

FIG. 15 illustrates an alopecia assessment in the ears of OXA-induced dermatitis mice comparing WT mice and GzmK−/− mice showing that alopecia is reduced in the GzmK−/− mice compared to WT controls.

FIG. 16 illustrates a combined severity score assessment in the ears of OXA-induced dermatitis mice comparing WT mice and GzmK−/− mice showing that overall severity was reduced in the GzmK−/− mice from d15 compared to WT controls.

FIG. 17 compares lesional coverage in the ears of OXA-induced dermatitis mice for WT mice and GzmK−/− mice measured from H&E stained ear tissue at d7, d17, and d27 showing reduced lesional severity for GzmK−/− mice compared to WT controls (data presented as the overall percentage of the ear surface covered in lesions).

FIGS. 18A and 18B compare GzmK immunohistochemistry in human pressure injury tissue showing GzmK+ cells elevated in human pressure injury tissue (18B) compared to control skin (18A).

FIGS. 19A and 19B compare GzmK immunohistochemistry (19B) and TBO (mast cell) (19A) sequential staining of human pressure injury tissue showing that a majority of mast cells express GzmK, but that other cell types also express GzmK.

FIG. 20 illustrates the pressure injury mouse model described and used herein.

FIGS. 21A and 21B compare GzmK immunohistochemistry in mouse pressure injury tissue showing increased number of GzmK+ cells at wound margin at d3 post-injury compared to unwounded controls.

FIG. 22 illustrates improved wound closure for WT mice and GzmK−/− mice as measured by wound margin in H&E stained tissue sections of mouse pressure injury tissue at d3, d7, and d10 post-injury. GzmK−/− mice displayed significantly increased wound margin (measured from the wound margin in the mid-point of the dermis) at d3 and d10 compared to WT mice.

FIG. 23 illustrates the results of an in vitro syndecan-1 cleavage assay. In the assay, recombinant syndecan-1 (0.7 ug) was incubated with recombinant GzmA (500 nM), GzmK (500 nM), and GzmB (500 nM), then run on a coomassie gel. Syndecan-1 in the absence of granzymes were included as a control. The results show that syndecan-1 was cleaved by all three granzymes.

FIGS. 24A and 24B illustrate syndecan-1 immunocytochemistry. HaCaTs were cultured to confluence, placed on FBS-free medium for 24 h, then GzmK-treated (0, 10 and 100 nM) for 14 h. Cells were fixed, blocked then incubated overnight with syndecan-1 antibody. Wells were washed then incubated for 1 h with anti-rabbit 488. DAPI was included as a nuclear stain. Images captured with fluorescence microscope (24A). Intensity was quantified using Image J (25B).

FIGS. 25A-25D illustrate syndecan-1 immunohistochemistry. Syndecan-1 was analyzed in human pressure injury tissue (25B, 25C, and 25D) and unwounded control skin (25A). The results show reduced syndecan-1 staining intensity in the pressure injury tissue samples (25B, 25C, and 25D).

FIGS. 26A-26C show that syndecan-1 was reduced in mouse tissue injury. Syndecan-1 was analyzed in mouse pressure injury tissue (d7) by immunohistochemistry. There is reduced syndecan-1 staining intensity in WT mice (26A) compared to GzmK−/− mice (26B) tissue samples. Quantitation of syndecan-1 reduction is compared in FIG. 26C.

FIGS. 27A-27D compare Prussian Blue staining in OXA-induced dermatitis ears at day 17 (27B) and day 27 (27C) to uninjured ear controls (27C) showing that staining is elevated in WT ears compared to GzmK−/− ears suggesting that GzmK has a role in vessel damage and hemostasis. Staining quantitation is shown in FIG. 27D.

FIGS. 28A and 28B compare GzmK immunohistochemistry of healthy (28A) and psoriasis-affected (28B) human skin (n=3) showing increased GzmK positive cells in psoriasis-affected tissue compared to healthy skin. GzmK positive cells appear to localize to the dermal immune cell infiltrate, predominantly in lymphocytes and cells with dendritic processes. The inset shows high magnification.

FIG. 29A shows a representative photographic comparison of drug-induced psoriasis in KO (GzmK−/−) mice and WT mice at day 0 and day 7 (n=6 per group). The black box represents the area where IMQ was applied and subsequently scored for severity.

FIG. 29B compares the daily change in skin severity in IMQ-treated WT and KO mice showing increased severity (defined as cumulative erythema and squamae scores) in WT mice compared to KO mice.

FIG. 30A shows representative hematoxylin and eosin stained dorsal tissue of untreated and IMQ-treated WT and untreated and IMQ-treated KO mice at day 7 (n=6 per group).

FIG. 30B is a multivariable linear regression with 3^{rd} degree interaction comparing epidermal thickness of dorsal tissue in untreated and IMQ-treated WT and untreated and IMQ-treated KO mice at day 7. Increased epidermal thickness in WT mice compared to KO mice.

FIG. 30C shows representative Ki67 immunohistochemistry of dorsal tissue in untreated and IMQ-treated WT and untreated and IMQ-treated KO mice at day 7.

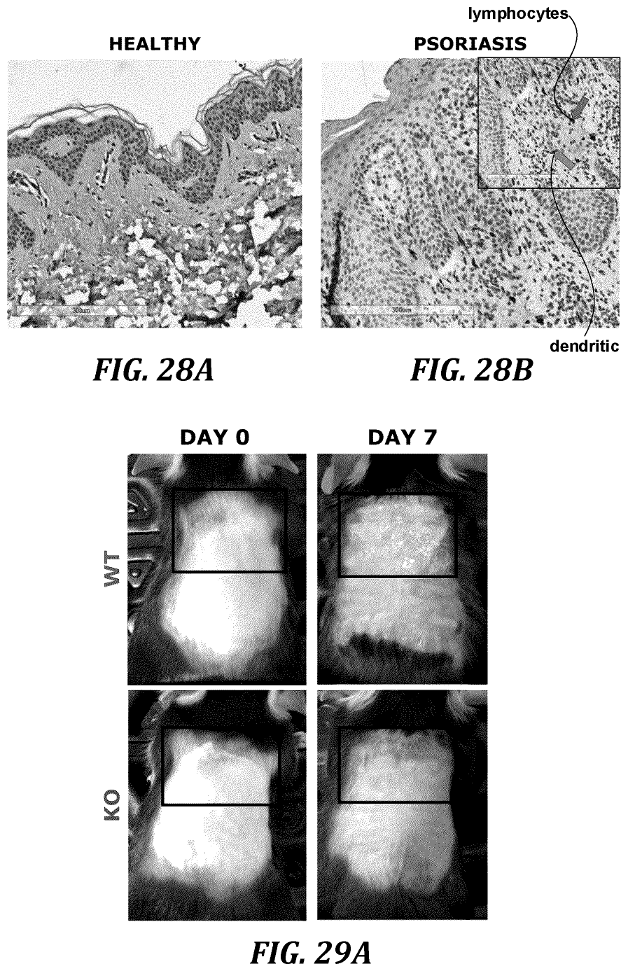

For FIGS. 28A-30C data are presented as mean±standard error of the mean (*P<0.05, **P<0.005, and ***P<0.001 compared with WT controls.

### DETAILED DESCRIPTION OF THE INVENTION

Granzyme K (GzmK) is elevated in tissues following wounding/cutaneous tissue injury and in response to inflammatory skin disease. This, in turn, has a negative effect on wound repair and regeneration. As described herein, reducing the activity of Granzyme K has a positive effect on wound repair and regeneration. Inhibition of Granzyme K may provide a therapeutic approach to treat these ailments.

The data described herein confirms that GzmK is indeed elevated in wounds, such as burns (human and mouse), and pressure injury (human and mouse), and inflammatory skin conditions, such as psoriasis (human) and atopic dermatitis (human), compared to healthy control skin.

In murine models of wound healing (burns and pressure injury), the presence of GzmK contributes to worsen wound severity compared to those mice without GzmK (i.e., GzmK knockout mice, GzmK−/− mice).

In murine models of inflammatory skin disease (psoriasis and atopic dermatitis), the presence of GzmK contributes to worsen disease severity compared to those mice without GzmK (i.e., GzmK knockout mice, GzmK−/−).

As described herein, GzmK impairs re-epithelialization (i.e., closure of the epidermis), an important step in wound repair as it provides a barrier against infection; GzmK cleaves syndecan-1 in keratinocytes, a major cell type of the epidermis, that functions to regulate cell migration and impairs wound healing when absent; and GzmK induces a pro-inflammatory response, including delays in the transition from a pro-inflammatory to a pro-healing wound repair phenotype.

Thus, the present invention provides methods for treating inflammatory skin conditions, treating skin wounds, and promoting skin wound healing by reducing the activity of Granzyme K.

In one aspect, the invention provides methods for treating inflammatory skin conditions, treating wounds, and promoting wound healing that involve reducing the activity of Granzyme K in a subject having an inflammatory skin condition or wound.

In one embodiment, the invention provides a method of treating an inflammatory skin condition (e.g., psoriasis or atopic dermatitis) in a subject, comprising reducing the activity of Granzyme K in a subject, thereby treating the inflammatory skin condition.

In another embodiment, the invention provides a method of treating a wound (e.g., a burn wound, chronic wound, acute wound, pressure injury, ischemic injury) in a subject, comprising reducing the activity of Granzyme K in a subject, thereby treating the wound.

In a further embodiment, the invention provides a method of promoting wound healing in a subject, comprising reducing pro-inflammatory cytokine response in keratinocytes, fibroblasts, and/or endothelial cells by reducing the activity of Granzyme K in the subject.

In yet another embodiment, the invention provides a method for promoting wound re-epithelization in a subject, comprising reducing the activity of Granzyme K (e.g., in keratinocytes proximate to the wound).

In yet a further embodiment, the invention provides a method of promoting wound healing in a subject, comprising inhibiting cleavage of syndecan-1 by reducing the activity of Granzyme K.

In another embodiment, the invention provides a method of preventing vascular permeability (leakage) in a subject, comprising Granzyme K-mediated immune cell recruitment and endothelial pro-inflammatory response in vessels located at the site of injury, by reducing the activity of Granzyme K.

In another aspect, the invention provides methods for treating inflammatory skin conditions, treating wounds, and promoting wound healing that involve inhibiting Granzyme K in a subject having an inflammatory skin condition or wound.

In one embodiment, the invention provides a method of treating an inflammatory skin condition (e.g., psoriasis or atopic dermatitis) in a subject, comprising administering an effective amount of Granzyme K inhibitor to the subject, thereby treating the inflammatory skin condition.

In another embodiment, the invention provides a method of treating a wound (e.g., a burn wound, chronic wound, acute wound, pressure injury, or ischemic injury) in a subject, comprising administering an effective amount of Granzyme K inhibitor to the subject, thereby treating the wound.

In a further embodiment, the invention provides a method of promoting wound healing in a subject, comprising inhibiting cleavage of syndecan-1 by administering an effective amount of Granzyme K inhibitor to the subject.

In yet another embodiment, the invention provides a method of promoting wound healing in a subject, comprising reducing pro-inflammatory cytokine response in keratinocytes, fibroblasts, macrophages, and/or endothelial cells by administering an effective amount of Granzyme K inhibitor to the subject.

In yet a further embodiment, the invention provides a method for promoting wound re-epithelization in a subject, comprising inhibiting cleavage of syndecan-1 in a keratinocyte by administering an effective amount of Granzyme K inhibitor to the subject.

In another embodiment, the invention provides a method for promoting wound re-epithelization in a subject, comprising administering an effective amount of Granzyme K inhibitor to the subject.

In a further aspect, the invention provides methods for converting a pro-inflammatory phenotype to a pro-healing wound repair phenotype.

In one embodiment, the invention provides a method of stimulating re-epithelialization, comprising inhibiting syndecan-1 cleavage in the keratinocyte by reducing the activity of Granzyme K in the wounded or damaged tissue area.

In another embodiment, the invention provides a method of converting a pro-inflammatory phenotype to a pro-healing wound repair phenotype, comprising reducing pro-inflammatory cytokine response in keratinocytes, fibroblasts, macrophages, and/or endothelial cells by reducing the activity of GzmK in the wounded or damaged tissue.

The effectiveness of the methods of the invention is described below.

### Wound Healing

In one aspect, the invention provides methods of treating a wound or promoting wound healing in a subject. The methods of the invention are suitable for treating or promoting the healing of wounds including burn wounds (thermal injury), chronic wounds, acute wounds, pressure and ischemic injury (e.g., ischemia reperfusion injury).

In certain embodiments, the methods include reducing the activity of Granzyme K in a subject, thereby treating the wound or promoting wound healing in the subject. In certain embodiments, the method includes administering an effective amount of a Granzyme K inhibitor to the subject, thereby treating the inflammatory skin condition in the subject.

Suitable Granzyme K inhibitors include small molecules (e.g., organic compounds having a molecule weight less than about 800 g/mole), nucleic acids, peptides, or proteins, such as antibodies. In one embodiment, the Granzyme K inhibitor is an inter-alpha inhibitor protein (IαIp). In another embodiment, the Granzyme K inhibitor is bikunin.

Thermal Injury

The role of Granzyme K (GzmK) in inflammation and remodeling in response to thermal injury is described herein. In human burn tissue, GzmK was found to be elevated compared to normal skin, with expression predominantly found in macrophages. GzmK was expressed and secreted by cultured human classically-activated macrophages. To assess the role of GzmK in response to skin wounding, wild-type (WT) and GzmK−/− mice were subjected to a grade 2 thermal injury. GzmK−/− mice exhibited improved wound closure, matrix organization and tensile strength compared to wild-type mice. Reduced pro-inflammatory IL-6, ICAM-1, VCAM-1, and MCP-1 expression was observed at 3 days post-injury. Additionally, GzmK induced IL-6 expression in keratinocytes and skin fibroblasts that was dependent on protease activated receptor-1 (PAR-1) activation. Re-epithelialization showed the greatest degree of improvement of all healing parameters, suggesting keratinocytes are sensitive to GzmK-mediated proteolysis. In support, keratinocytes, but not skin fibroblasts, exposed to GzmK demonstrated impaired wound healing in vitro. In summary, GzmK influences wound healing by augmenting inflammation while impeding epithelialization.

GzmK Elevated in Human Burns and Secreted by Classically Activated Macrophages

GzmK expression was evaluated in human acute burn tissues excised from day (d) 2 to d30 post-injury. See Table 1.

TABLE 1
Burn Wound Patient Data.

| | Days | | | |
|---|---|---|---|---|
| | (post- | | | |
| Sample | wound) | Patient information | Injury site | Notes |
| 1 | | | Hand | |
| 2 | 21 | 34 years, male | Right forearm | Smoker |
| 3 | 14 | 42 years, male | Trunk | Smoker |
| 4 | 10 | 25 years, female | Buttock | Smoker |
| 5 | 30 | 44 years, male, Caucasian | Right leg | Smoker |
| 6 | 2 | 21 years, male, Caucasian | Right hand | Smoker |
| 7 | 6 | 23 years, male, Caucasian | Right forearm | Smoker |
| 8 | 3 | 21 years, male, Caucasian | Trunk | Smoker |
| 9 | 5 | 34 years, male, Caucasian | Trunk | — |

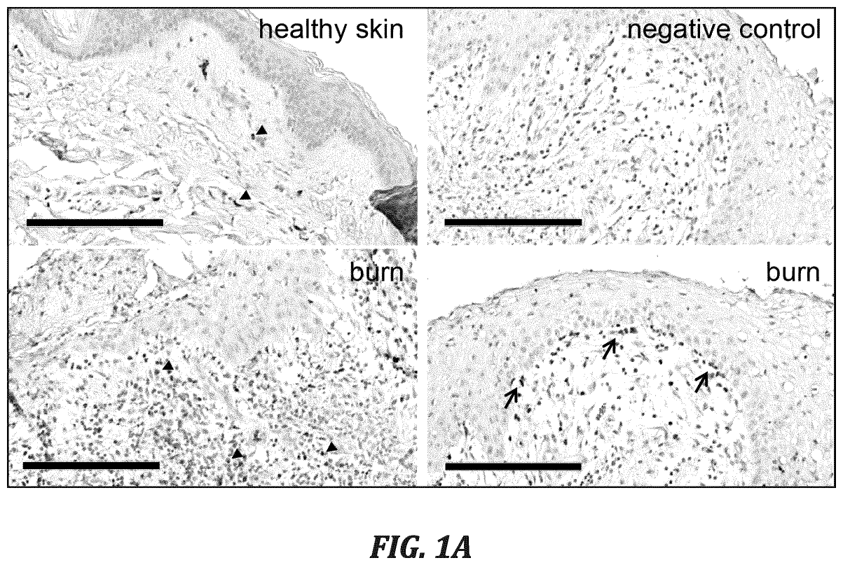

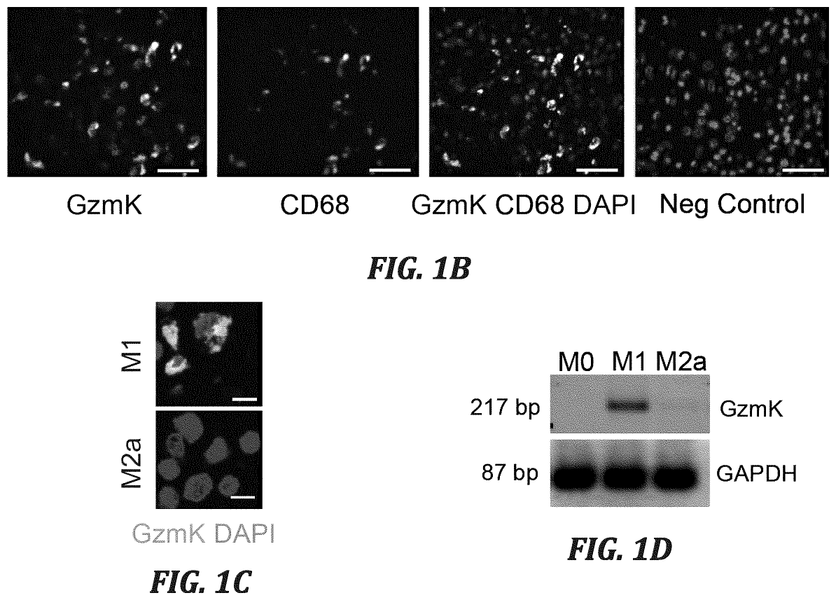

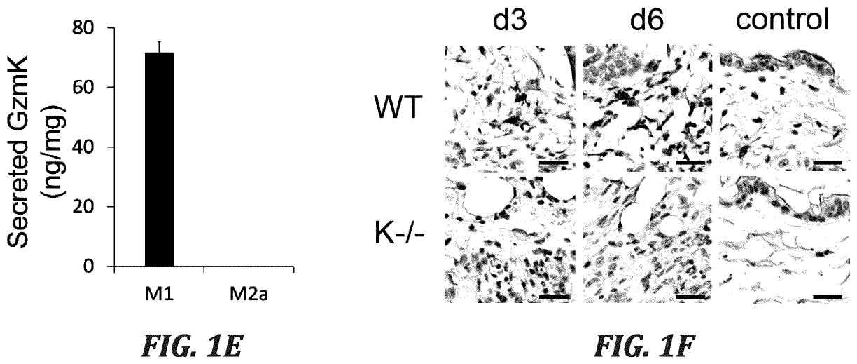

In healthy undamaged skin, GzmK+ cells were minimally dispersed throughout the dermis (FIG. 1A). In contrast, partial thickness burn injured skin exhibited increased numbers of GzmK+ cells, with the vast majority localized to the inflammatory cell infiltrate, but also in close proximity to the dermal-epidermal junction. Notably, the amount and localization of GzmK+ cells was similar between all nine burn samples, despite differences in time post-injury, wound location and wound severity.

GzmK strongly co-localized with CD68+ cells (marker of circulating monocyte and tissue macrophages) within burn tissue (FIG. 1B). A separate GzmK+ cell population was also observed in the burn wound tissue (FIG. 1B), albeit with reduced GzmK staining intensity. This cell population was not identified.

Within differing stages of wound repair, multiple polarized sub-types of macrophages have been identified, each performing unique roles in inflammation, including pro-inflammation (M1) and pro-healing (M2) (Murray, 2017). The macrophage sub-type(s) responsible for GzmK expression was therefore investigated. M1 macrophages exhibited GzmK immune-positivity, with negligible staining observed in M2a macrophages (FIG. 1C). Only classically activated M1 macrophages expressed GzmK mRNA (FIG. 1D), supporting the immune-fluorescence. GzmK secretion was also markedly elevated in M1 macrophages, whilst negligible levels were released by M2a macrophages (FIG. 1E).

Improved Wound Healing in GzmK−/− Mice

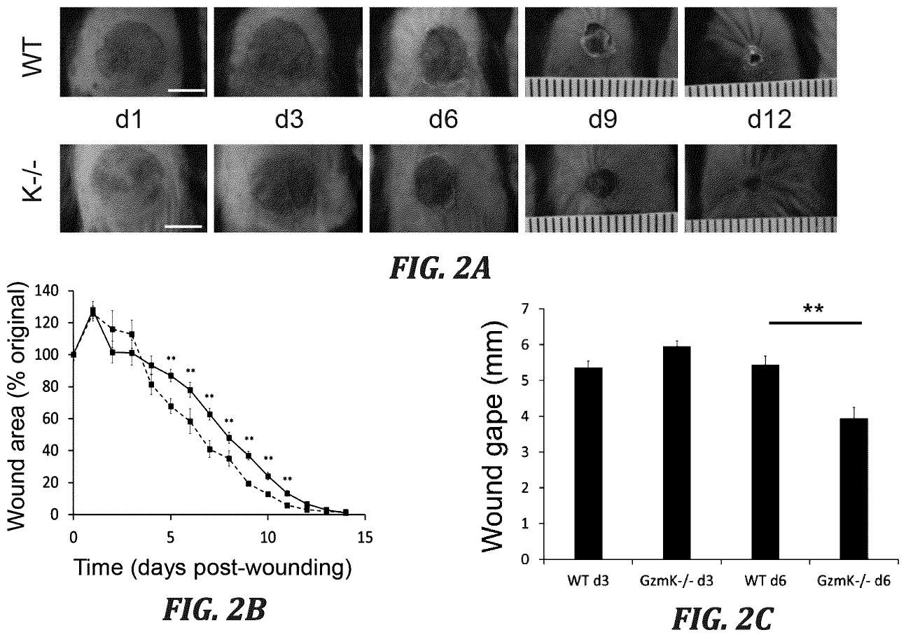

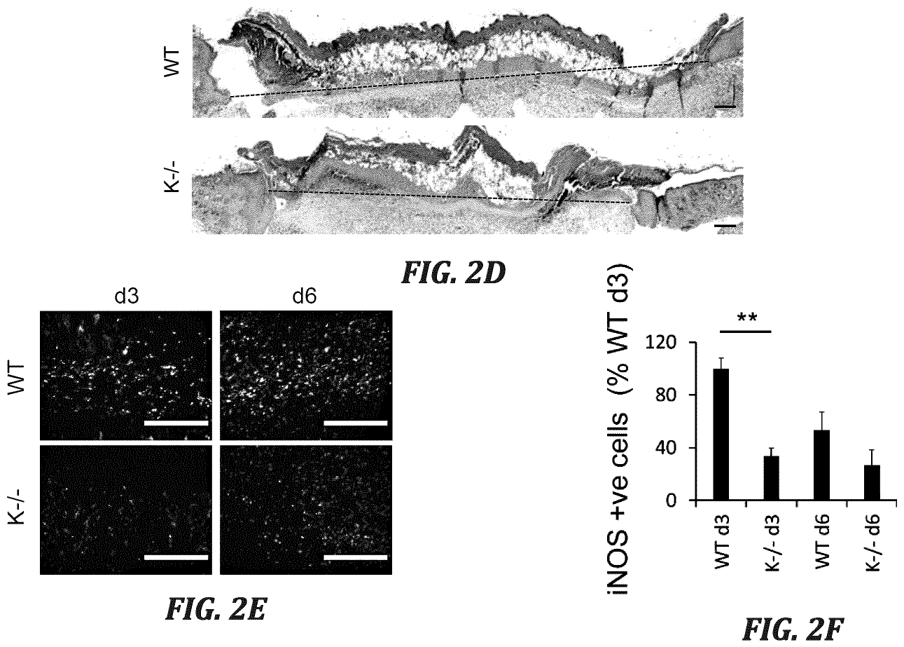

GzmK−/− and wild-type (WT) mice were subjected to thermal injury on the dorsum of 8 week old female mice. Wounds were partial thickness (grade 2b) as shown by tissue damage penetrating into the dermis but not the muscle layer (FIG. 2D), as reported previously (Shen et al., 2012).

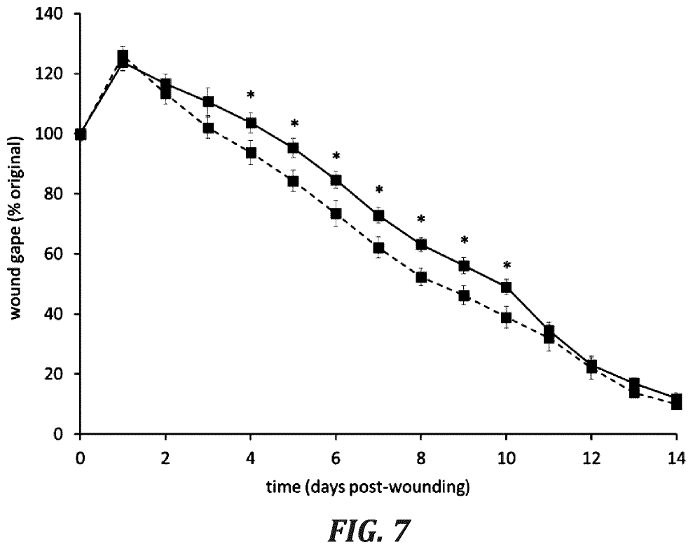

Negligible GzmK immune-reactivity was evident in healthy control skin, but GzmK+ cells were detected in WT mouse burns at both d3 and d6; localizing to the inflammatory cell infiltrate at the wound margin (FIG. 2E). GzmK immune-reactivity was absent in GzmK−/− mice burns. Macroscopically, there was a significant reduction in both wound area (P<0.005; FIGS. 2A and 2B) and wound gape (P<0.05; FIG. 7) from d5 until d10 post-injury in GzmK−/− compared to WT mice. Histological assessment supported the macroscopic data, showing a significant reduction in wound gape at d6 (P<0.005; FIGS. 2C and 2D).

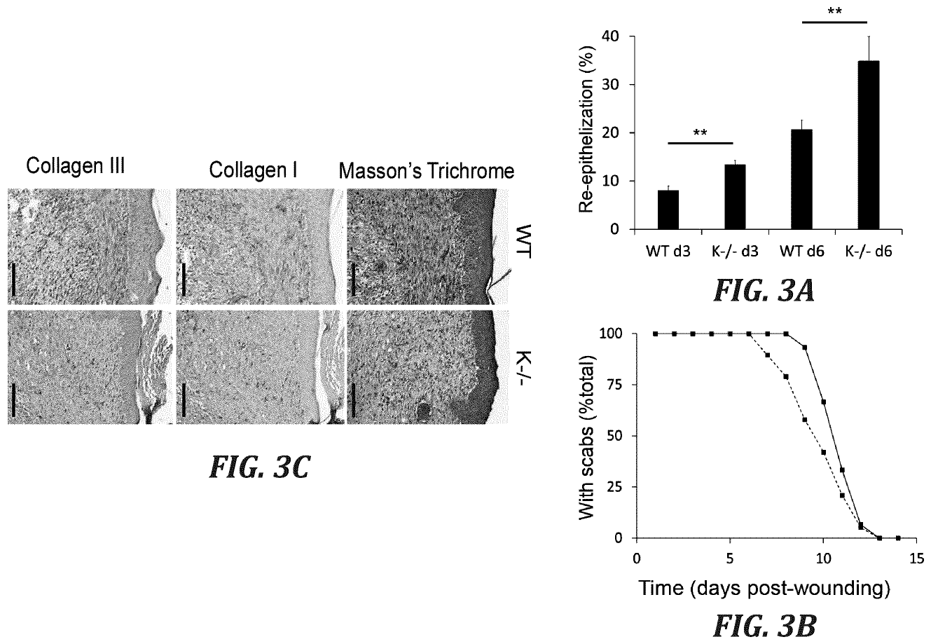

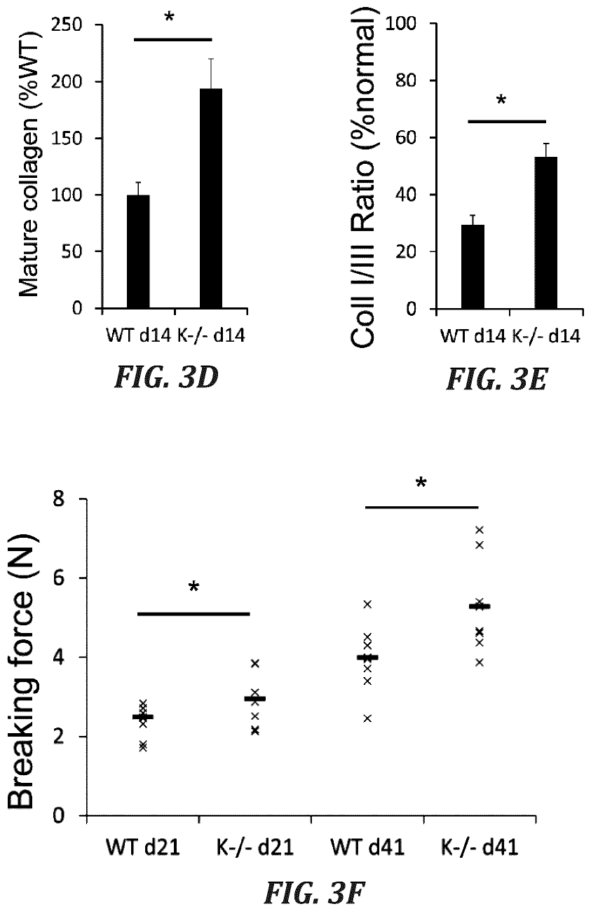

Re-epithelialization post-injury was significantly improved in GzmK−/− mice at both d3 and d6 compared to WT mice (P<0.005; FIG. 3A). Supporting enhanced re-epithelialization, scabs were observed to drop off GzmK−/− mice wounds approximately two days (25%) earlier than WT controls (FIG. 3B).

Masson's Trichrome staining of GzmK−/− burn wounds at d14 post-injury showed improved collagen maturation within the wounded dermal area compared to those in WT mice (P<0.05; FIGS. 3C and 3D). The Collagen-I to Collagen-III ratio was also significantly elevated in GzmK−/− wounds compared to WT (P<0.05; FIGS. 3C and 3E). The tensile strength of wounds showed improvement at both days 21 (P=0.046) and 41 post-injury (P=0.026) compared to WT controls (FIG. 3F).

GzmK Impairs Healing of Wounded Keratinocytes

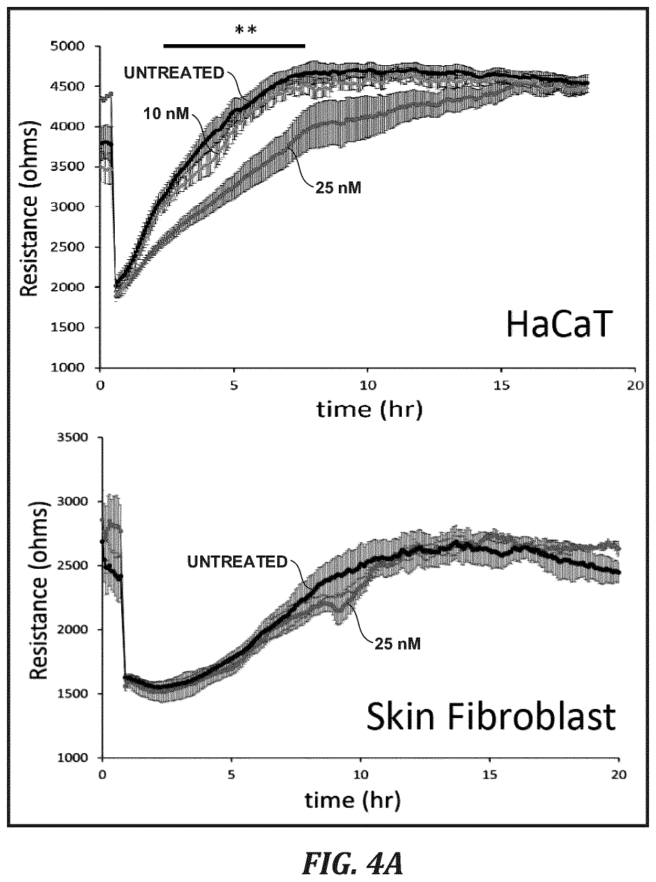

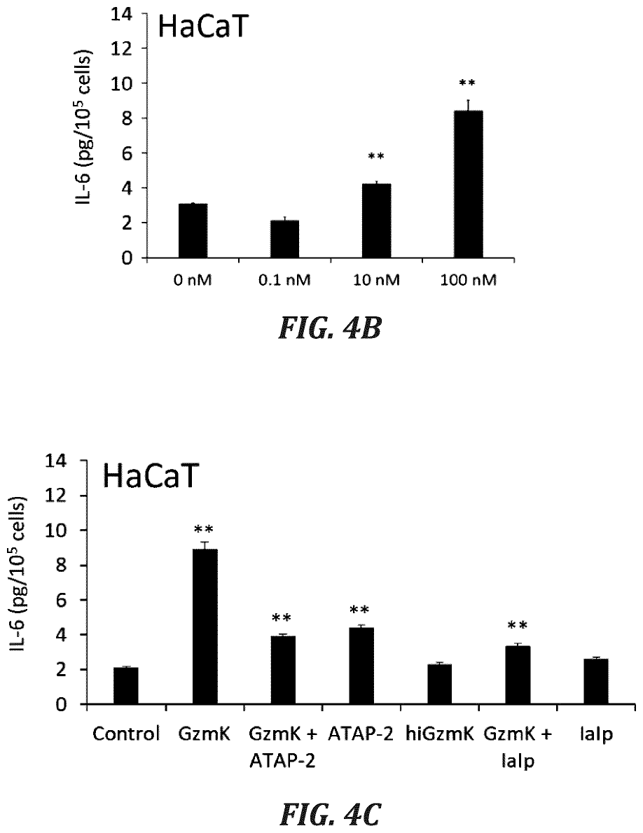

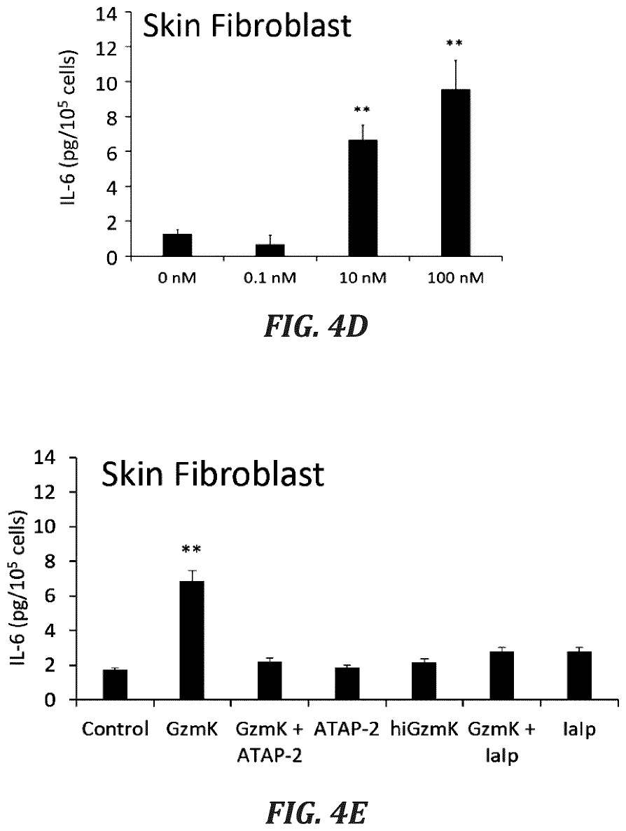

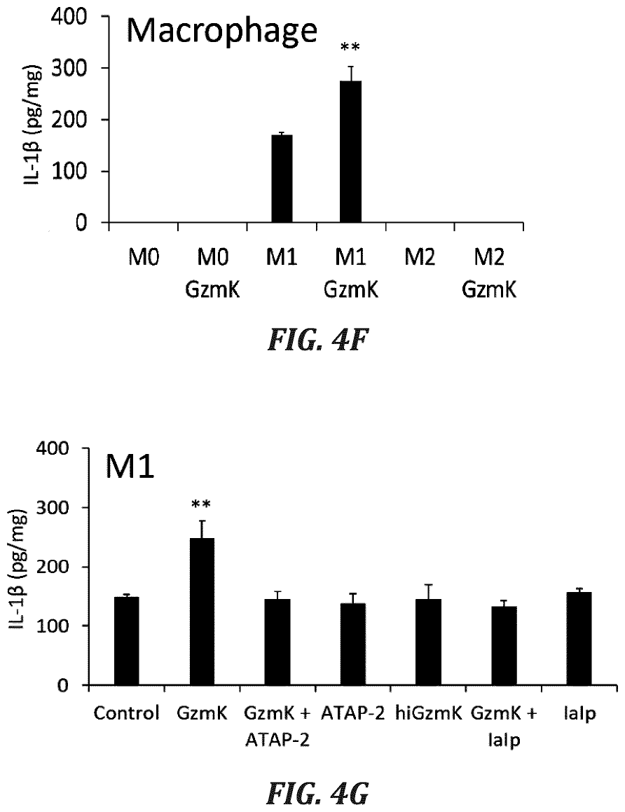

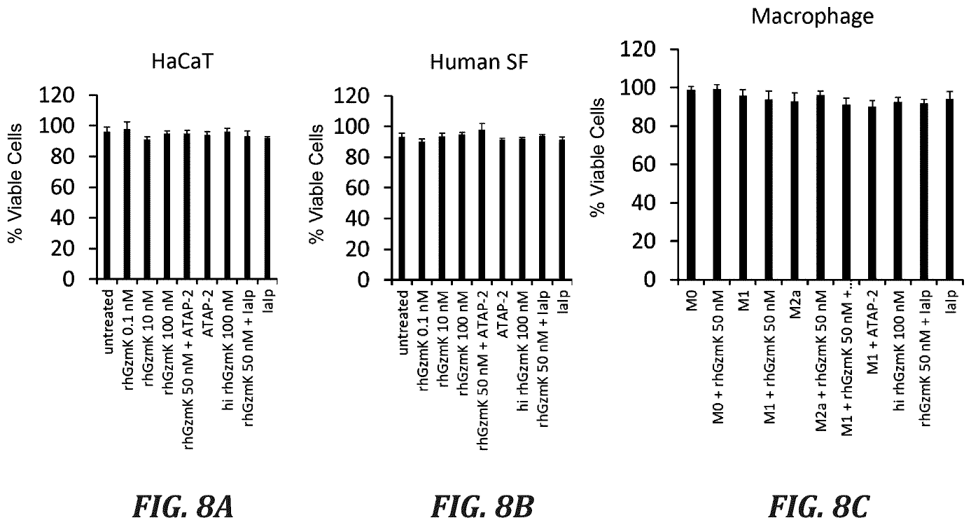

As classically activated macrophages secrete GzmK (FIG. 1E), the downstream effects of GzmK were investigated in vitro in human HaCaTs (keratinocytes) and primary human skin fibroblasts, the predominating cell types in skin. Addition of recombinant human GzmK (rhGzmK) to cells (≤100 nM) showed no detectable cytotoxicity up to 48 hours in culture (FIG. 8), as previously reported in endothelial cells (Sharma et al., 2016) and lung fibroblasts (Cooper et al., 2011). Using an Electric Cell-substrate Impedance Sensing (ECIS) wound healing assay, rhGzmK exhibited a dose-dependent and reproducible impairment of wound closure in HaCaTs, which was approximately 50% slower compared to untreated controls (FIG. 4A). Improved HaCaT migration in the absence of GzmK may help explain the improved re-epithelialization observed in GzmK−/− compared to WT mice burns. In contrast to HaCaTs, skin fibroblasts showed no change in wound closure in response to rhGzmK.

GzmK Induces PAR-1 Mediated Pro-Inflammatory Cytokine Release from Keratinocytes, Skin Fibroblasts and Classically Activated Macrophages

rhGzmK induces pro-inflammatory cytokine expression in both endothelial cells and lung fibroblasts, functioning through a PAR-1-mediated pathway (Cooper et al., 2011, Sharma et al., 2016). Studies were therefore performed to determine whether GzmK exposure in HaCaTs and skin fibroblasts induced pro-inflammatory cytokine expression in a similar fashion, thus providing mechanistic details regarding GzmKs role in burn wound repair. rhGzmK significantly increased IL-6 secretion from both HaCaTs (P<0.005 at ≥10 nM, FIG. 4B) and skin fibroblasts (P<0.005 at ≥10 nM, FIG. 4D) in a dose-dependent manner, with each secreting similar amounts. When cells were pre-incubated for 30 minutes with ATAP-2 (5 μg/mL), a PAR-1 neutralizing antibody, prior to rhGzmK (50 nM) treatment, significant reductions in IL-6 secretion from HaCaTs (P<0.005, FIG. 4C) and a complete amelioration of GzmK-mediated skin fibroblast IL-6 secretion (P<0.005, FIG. 4E) was observed compared to untreated controls.

GzmK induces LPS-activated primary mouse macrophages to process and secrete the pro-inflammatory cytokine, IL-1β (Joeckel et al., 2011). As described herein, THP-1-derived M0, M1 and M2a macrophages were exposed to rhGzmK in the absence of perforin and IL-1β secretion was determined. Cells treated with up to 100 nM rhGzmK showed no evidence of cytotoxicity (FIG. 8). Untreated M1 secreted IL-1β, whilst incubation with rhGzmK (50 nM) significantly increased IL-1β release (P<0.005, FIG. 4F). M0 or M2a did not release IL-1β with or without exposure to rhGzmK. Pre-incubation of M1 macrophages with the PAR-1 antagonist ATAP-2 (5 μg/mL) prior to the addition of rhGzmK (50 nM) ameliorated the GzmK-mediated release of IL-1β (FIG. 4G), suggesting GzmK-mediated IL-1β secretion from macrophages to be PAR-1-dependent. No effect on IL-1β secretion in response to pre-incubation of cells with ATAP-2 alone was observed.

GzmK Augments Pro-Inflammatory Cytokine Expression in Mice Burn Wounds

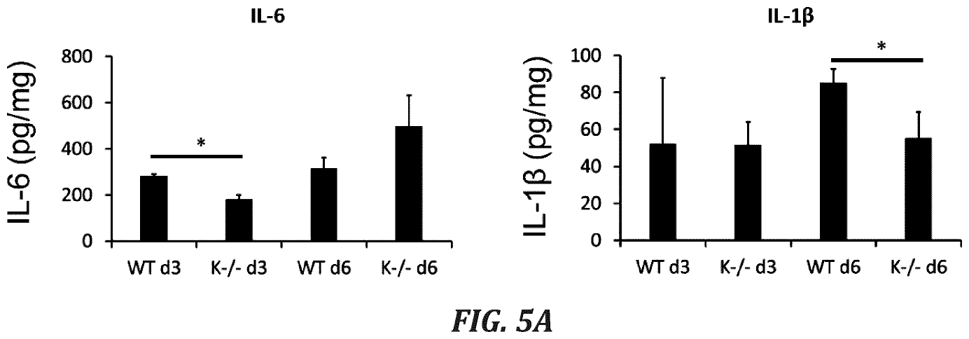

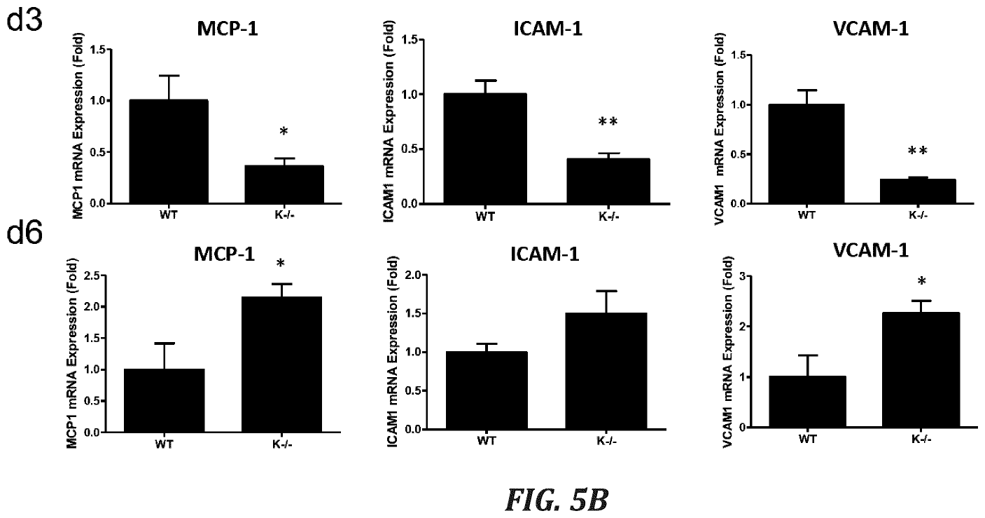

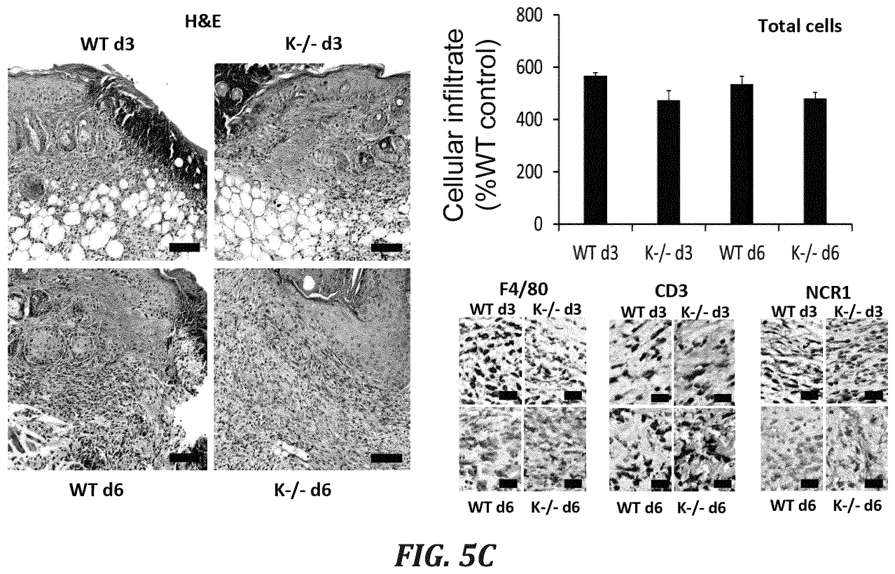

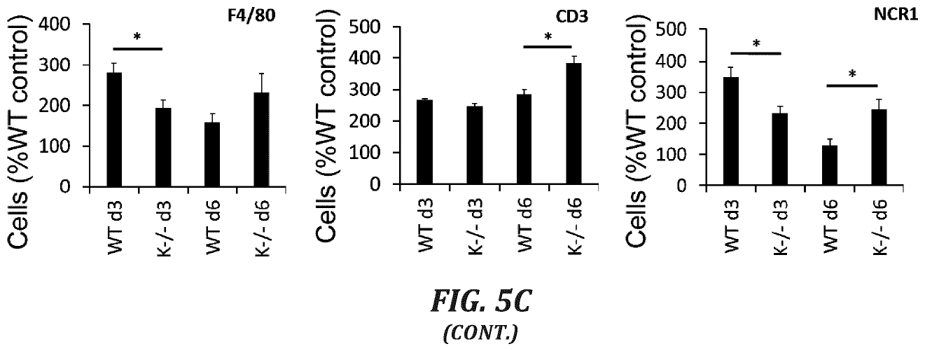

GzmK−/− mice wounds at d3 post-injury showed significantly reduced IL-6 protein compared to WT controls (P<0.05; FIG. 5A). At d6 post-injury, this pattern was reversed, trending to increased IL-6 protein (P=0.062). IL-6 protein concentration showed no difference at d3, but was significantly reduced in GzmK−/− mice burns at d6 post-injury (P=0.014).

Augmented Chemokine and Adhesion Molecule Expression in GzmK−/− Mice Burn Wounds

Endothelial cells cultured with rhGzmK increase MCP-1, ICAM-1, and VCAM-1 expression (Sharma et al., 2016), thus gene expression of each was quantified in mouse burn wounds. MCP-1 (P=0.035), ICAM-1 (P=0.0049) and VCAM-1 (P=0.0017) expression in GzmK−/− mice at d3 post-injury were significantly reduced compared to WT mice (FIG. 5B). At d6, and opposite to the pattern observed at d3, MCP-1 (P=0.025), ICAM-1 (non-significant, P=0.2) and VCAM-1 (P=0.023) expression were increased in GzmK−/− mice.

GzmK Increases Macrophage Recruitment to Mice Burn Wounds

No difference in the amount of inflammatory cell infiltrate was detected between GzmK−/− and WT mice at both d3 and d6 post-injury (FIG. 5C). However, macrophage/monocyte and NK cell numbers were significantly reduced in GzmK−/− mice at d3 post-injury compared to WT burns (P<0.05), whilst no change was evident in T-cells. In contrast, at d6, GzmK−/− mice showed an increase in T-cells (P<0.05), NK cells (P<0.05) and macrophages/monocytes (not significant, P=0.12) compared to WT mice. As M1 are the predominant macrophage sub-type expressing GzmK in human burns, iNOS positive cells were therefore quantified in mice burns. The number of M1 macrophages was reduced in GzmK−/− compared to WT burns at d3 (P<0.005) and d6 (non-significant) post-injury (FIGS. 2E and 2F).

Non-fatal burns are a major cause of morbidity, leading to prolonged hospitalization, disfigurement, and disability. In the US alone, greater than 400,000 burn injuries occur each year, with approximately 20,000 of those requiring hospitalization (Peck, 2011). Limited therapeutic options are available. Consequently, new targeted strategies are required. Reducing the magnitude of inflammation immediately post-injury has been identified as one such target (Farina et al., 2013).

The present invention demonstrates for the first time that GzmK is abundant in burn wounds and plays a pathogenic role in inflammation, epithelialization and remodeling. Previously, GzmK expression was reported in CTLs, NK and CD4+ T-cells (Joeckel et al., 2017, Joeckel et al., 2011, Joeckel et al., 2012, Wilson et al., 2017). As described herein, in burn wounds, GzmK is predominantly localized to the CD68+ monocyte/macrophage cell populations within the dermis. Differentially polarized, pro-inflammatory/pro-reparative macrophages have been described, with both anti-inflammatory and pro-inflammatory cytokine expression reported to be induced simultaneously at early time-points during tissue repair (Murray, 2017). As described herein, classically activated M1 macrophages expressed and secreted GzmK, whilst M2a macrophages exhibited negligible GzmK expression. Thus, without being bound to theory, GzmK may contribute to the pro-inflammatory response following burn injury.

Thermal injury in GzmK−/− mice exhibited improved overall wound healing, enhanced re-epithelialization, improved dermal maturation and stronger tensile strength compared to WT mice wounds. Re-epithelialization was particularly striking in GzmK−/− compared to WT mice burns. The epithelial tongue in GzmK−/− mice exceeded double the length of those in WT mice as early as d3 post-injury. In vitro, GzmK impaired keratinocyte wound closure, suggesting a direct effect on cellular migration. Rapid re-epithelialization and wound closure greatly benefits overall wound healing, in part by re-establishing a barrier against infection; a major contributor to wounds transitioning into chronicity. The down side of increasing cell proliferation/migration during wound repair is the potential to induce fibrosis. The GzmK-mediated reduction in cell migration described herein, however, was limited to cultured keratinocytes, whereas fibroblasts, the major cell-type involved in fibrosis, showed no alteration in response to GzmK exposure.

Pro-inflammatory IL-6, essential for timely wound healing, is involved in generating acute phase responses, inflammation and lymphocyte differentiation (McFarland-Mancini et al., 2010). GzmK-mediated IL-6 secretion occurs in endothelial cells (Sharma et al., 2016), and our data showed GzmK-mediated IL-6 secretion from cultured HaCaTs and skin fibroblasts, releasing similar quantities from each, and both operating through PAR-1. Indeed, in GzmK−/− mice burns at d3 post-injury, IL-6 expression was reduced compared to equivalent WT samples. This trend appeared to be reversed by d6, suggesting the absence of GzmK may contribute to a delayed pro-inflammatory profile in response to thermal injury.

Joeckel et al., 2011 previously reported GzmK-mediated IL-1β secretion from LPS activated macrophages, with this predicted to be inflammasome-dependent (Joeckel et al., 2011). The data confirmed GzmK-mediated IL-1β secretion from classically activated macrophages, showing PAR-1 dependent release. As IL-1β has an important role in wound healing, providing a positive feedback loop capable of sustaining a persistent pro-inflammatory wound phenotype (Mirza et al., 2013), the effect of GzmK knockout on IL-1β expression post-thermal injury was investigated. Although there was no difference at d3 post-injury, IL-1β expression was significantly reduced in GzmK−/− burn wounds at d6. GzmK therefore appears to play a role in either delaying or possibly reducing the pro-inflammatory IL-1β profile, which may in turn decrease macrophage recruitment post-burn injury.

GzmK induces MCP-1, ICAM-1, and VCAM-1 expression in endothelial cells (Sharma et al., 2016), with these factors together facilitating immune cell adhesion and trans-endothelial migration (Ley et al., 2007). GzmK increased adhesion of THP-1 monocytes to cultured endothelial cells (Sharma et al., 2016) suggesting GzmK may directly affect immune cell recruitment. As described herein, MCP-1, ICAM-1, and VCAM-1 gene expression were significantly reduced at d3 post-injury in GzmK−/− compared to WT mice wounds, corresponding to a reduction in both macrophages and NK cells within the wound environment. Previously, GzmK−/− mice infected with Chikungunya virus, showing a significant reduction in foot swelling, had no overall change in inflammatory cell infiltrate or macrophage numbers, but NK and T-cells were both reduced (Wilson et al., 2017). The d3 post-injury data described herein also showed no change in overall inflammatory cell or T-cell recruitment and a reduction in NK cells. The reason different macrophage recruitment between these studies remains unknown, but can be explained by differences in the pathologies. At d6 post-injury, the pattern of MCP-1, ICAM-1, and VCAM-1 expression was reversed to that seen at d3, showing an increase in the GzmK−/− mice, and suggesting the pro-inflammatory response is not reduced but rather delayed post burn injury, and agreeing with the pro-inflammatory IL-6 expression data.

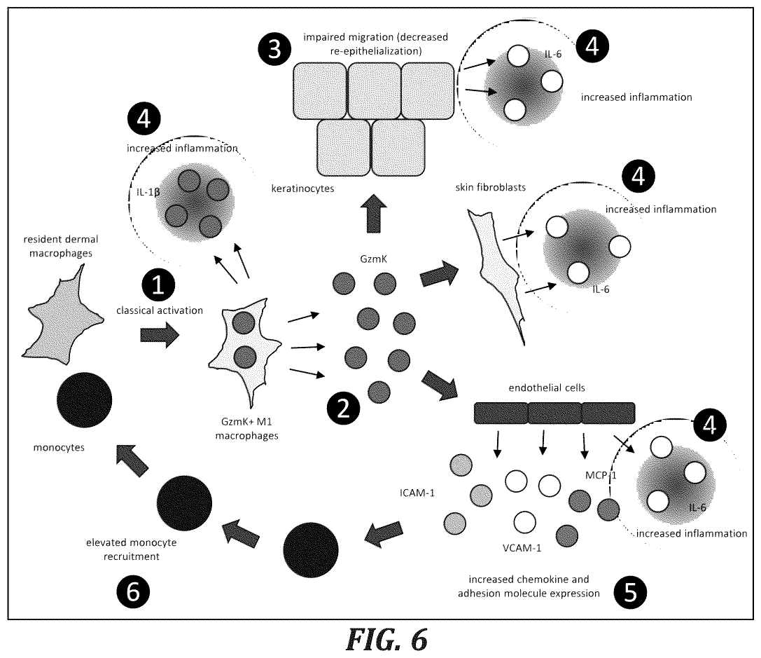

In conclusion, GzmK delays burn wound healing by impairing re-epithelialization, while promoting pro-inflammatory cytokine expression and subsequent immune cell recruitment to the site of injury (FIG. 6). As described herein, GzmK can be targeted to attenuate inflammation and promote epithelialization in the context of burn injury.

Pressure Injury

The methods of the invention are also useful in treating pressure injury. Inflammation associated with ischemia-reperfusion is a major contributor to pressure injury.

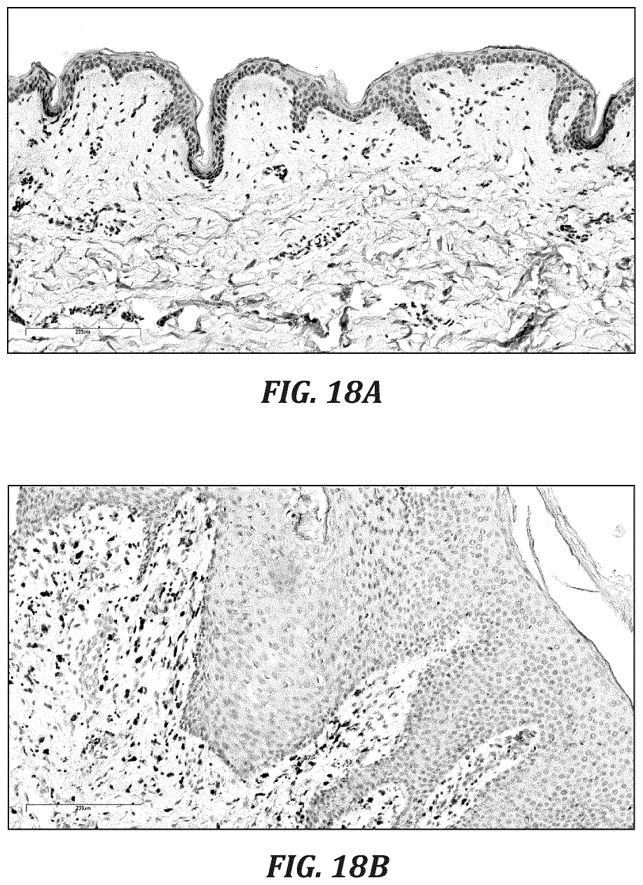

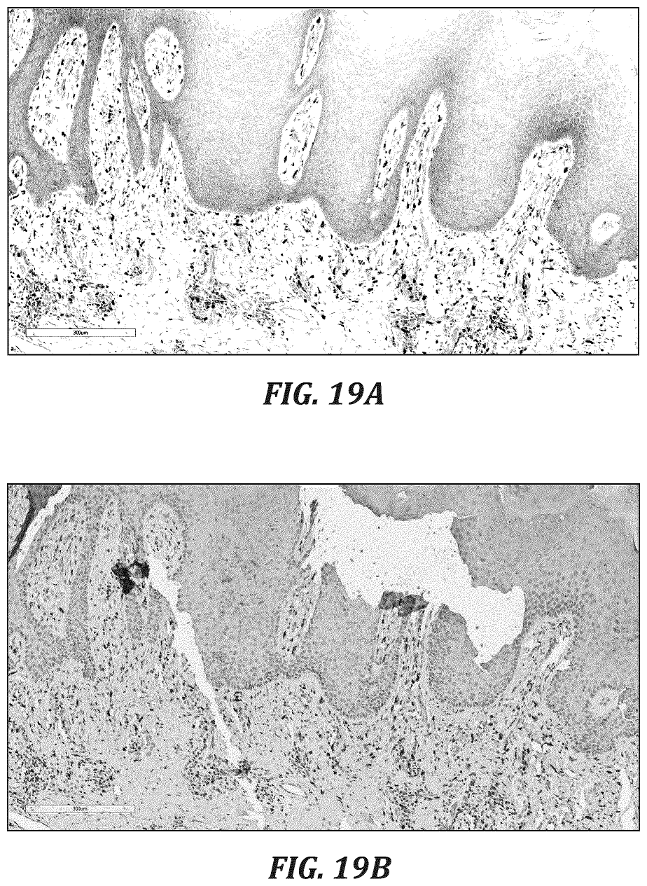

GzmK is elevated in pressure-injured tissues. FIGS. 18A and 18B compare GzmK immunohistochemistry in human pressure injury tissue showing GzmK+ cells elevated in human pressure injury tissue (18B) compared to control skin (18A). FIGS. 19A and 19B compare GzmK immunohistochemistry (19B) and TBO (mast cell) (19A) sequential staining of human pressure injury tissue showing that a majority of mast cells express GzmK, but other cell types also express GzmK.

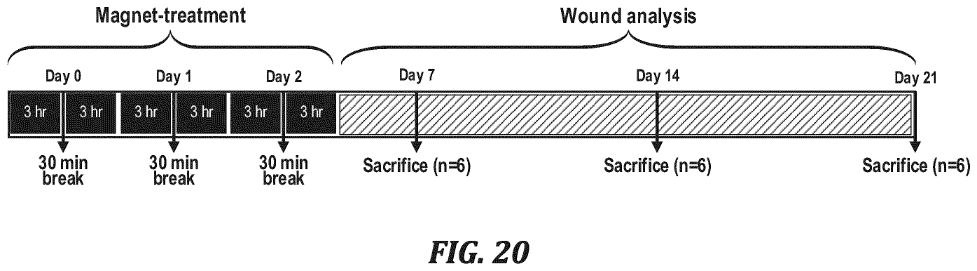

The pressure injury mouse model described and used herein is illustrated in FIG. 20.

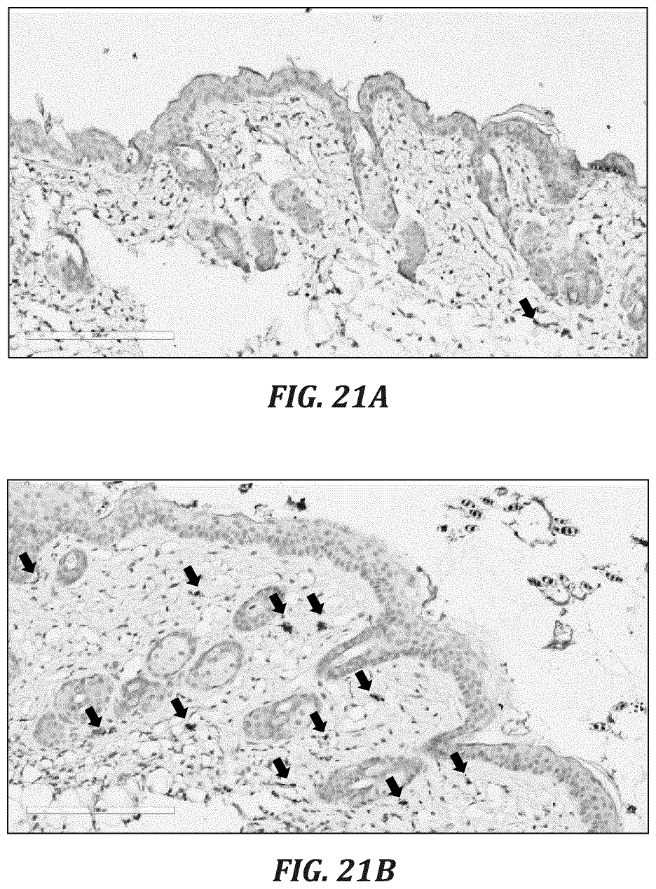

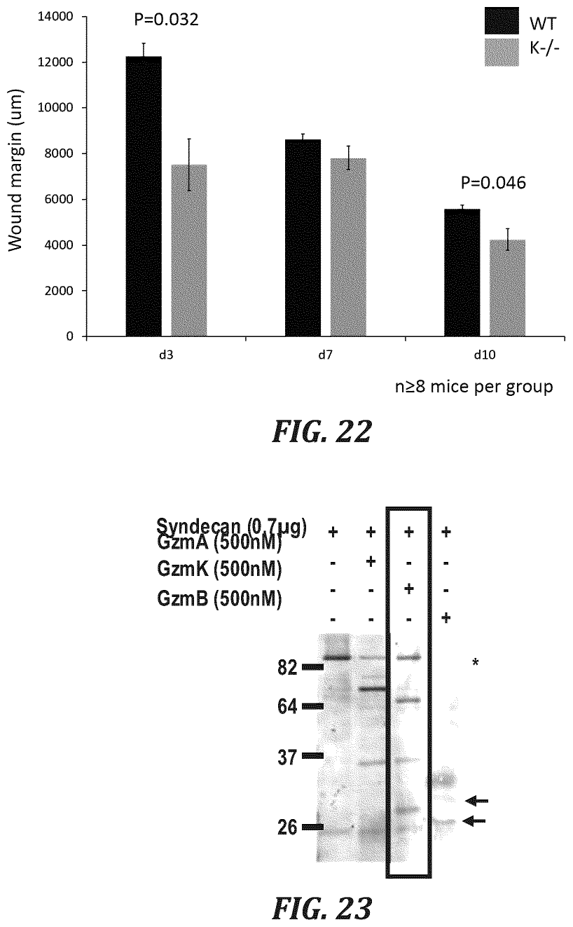

FIGS. 21A and 21B illustrate GzmK immunohistochemistry for mouse pressure injury tissue showing increased number of GzmK+ cells at wound margin at d3 post-injury compared to unwounded controls. FIG. 22 illustrates improved wound closure for WT mice and GzmK−/− mice as measured by wound margin in H&E stained tissue sections of mouse pressure injury tissue at d3, d7, and d10 post-injury. GzmK−/− mice displayed significantly increased wound margin at d3 and d10 compared to WT mice.

Syndecan-1 Promotes Wound Healing

Syndecan-1 is an integral membrane HS proteoglycan having a structure that allows binding with cytosolic, transmembrane, and extracellular matrix (ECM) proteins. Syndecan-1 plays important roles in mediating key events during wound healing because it regulates a number of important processes, including cell adhesion, cell migration, endocytosis, exosome formation, and fibrosis. Absence of syndecan-1 leads to delayed wound healing and increased neutrophil recruitment.

FIG. 23 illustrates the results of an in vitro syndecan-1 cleavage assay. In the assay, recombinant syndecan-1 (0.7 ug) was incubated with recombinant GzmA (500 nM), GzmK (500 nM), and GzmB (500 nM), then run on a coomassie gel. Syndecan-1 in the absence of granzymes were included as a control. The results show that syndecan-1 was cleaved by all three granzymes.

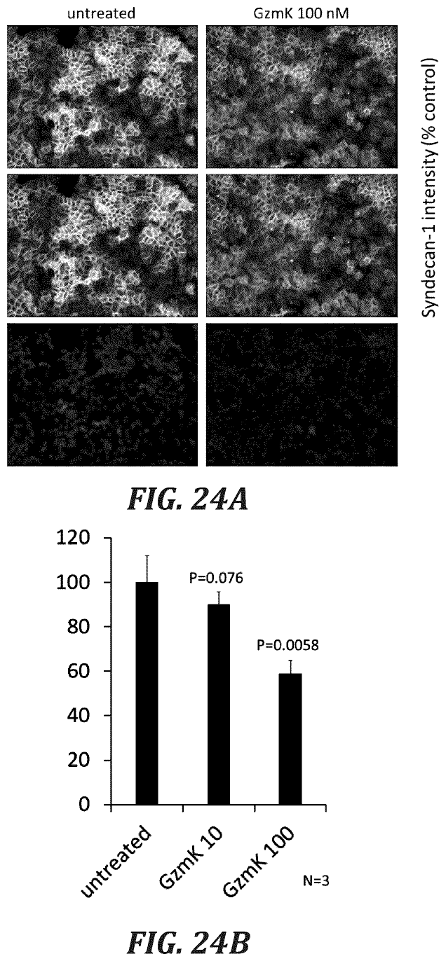

FIGS. 24A and 24B illustrate syndecan-1 immunocytochemistry. HaCaTs were cultured to confluence, placed on FBS-free medium for 24 h, then GzmK-treated (0, 10 and 100 nM) for 14 h. Cells were fixed, blocked then incubated overnight with syndecan-1 antibody. Wells were washed then incubated for 1 h with anti-rabbit 488. DAPI was included as a nuclear stain. Images captured with fluorescence microscope (24A). Intensity was quantified using Image J (25B).

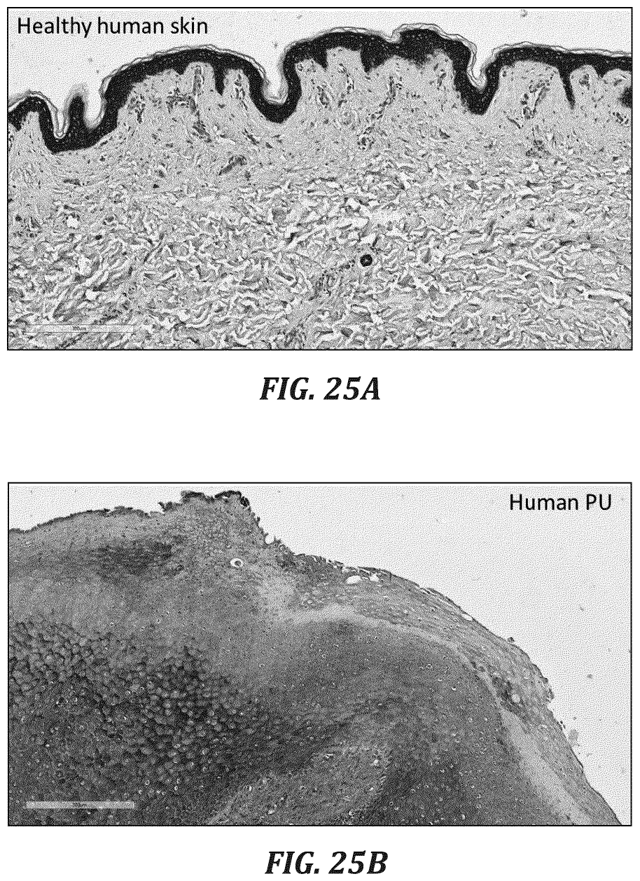

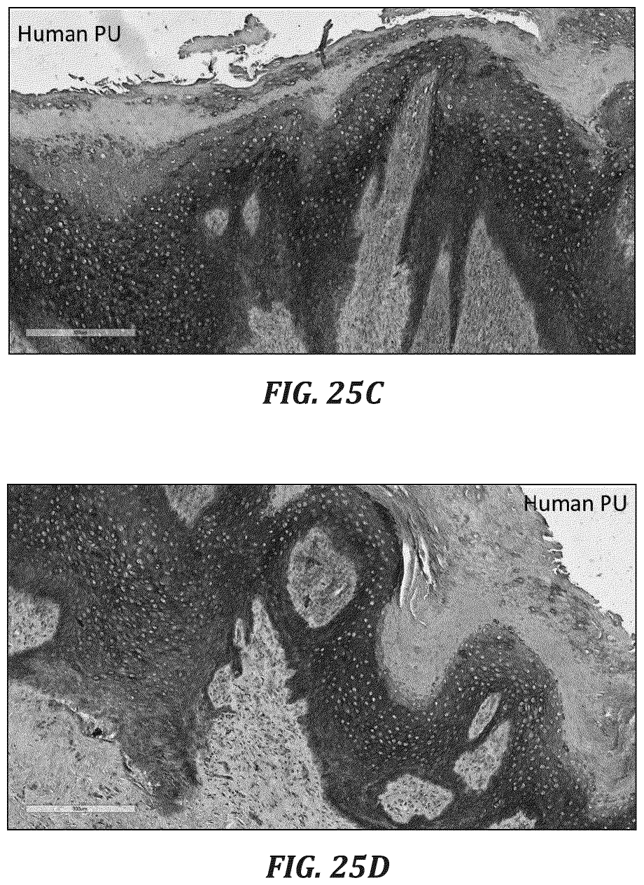

FIGS. 25A-25D illustrate syndecan-1 immunohistochemistry. Syndecan-1 was analyzed in human pressure injury tissue (25B, 25C, and 25D) and unwounded control skin (25A). The results show reduced syndecan-1 staining intensity in the pressure injury tissue samples (25B, 25C, and 25D).

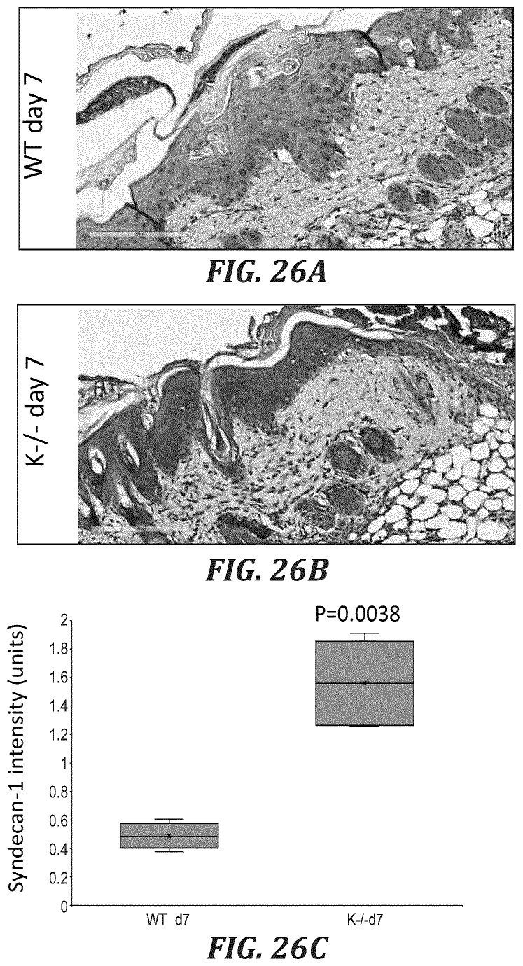

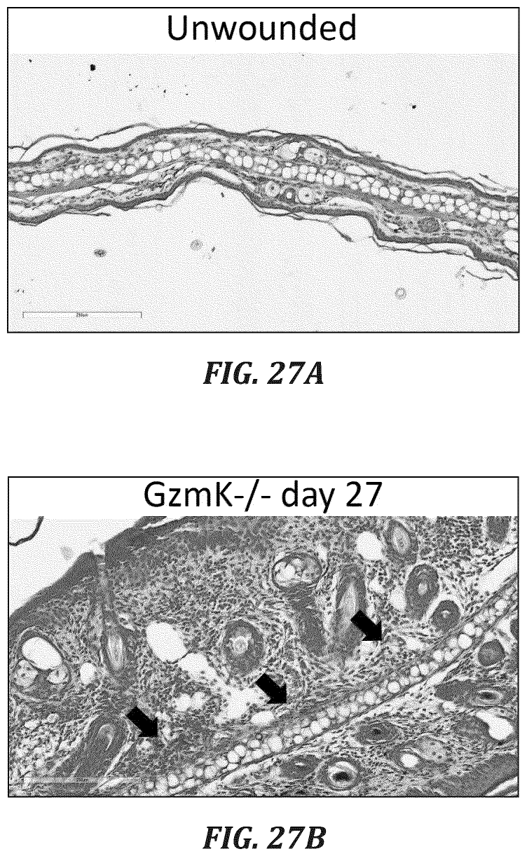

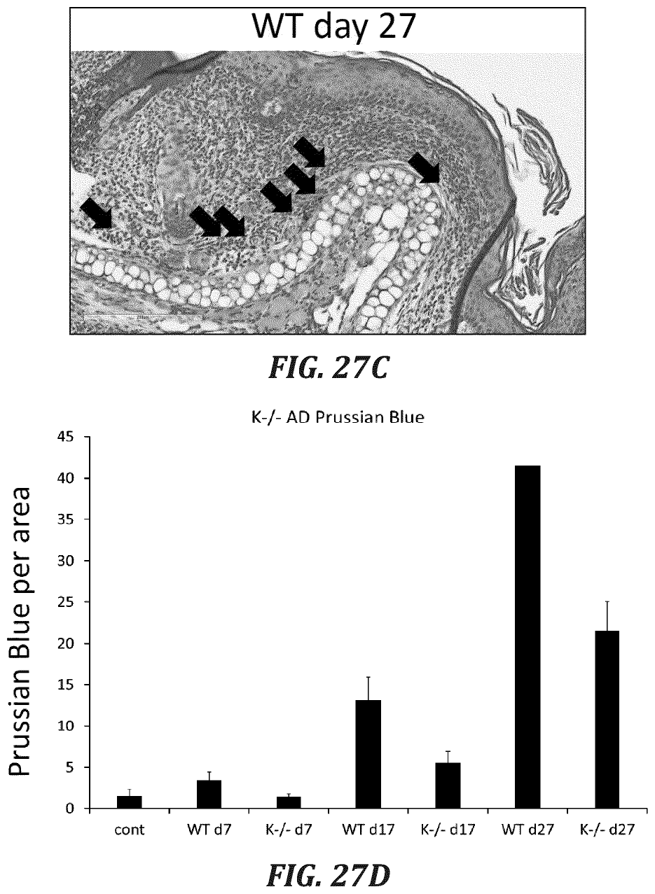

FIGS. 26A-26C show that syndecan-1 was reduced in mouse tissue injury. Syndecan-1 was analyzed in mouse pressure injury tissue (d7) by immunohistochemistry. There is reduced syndecan-1 staining intensity in WT mice (26A) compared to GzmK−/− mice (26B) tissue samples. Quantitation of syndecan-1 reduction is compared in FIG. 26C.

The methods of the invention are demonstrated to be effective in the treatment of wounds, including thermal and pressure wounds, where Granzyme K is elevated in the involved tissues.

### Inflammatory Skin Conditions

In another aspect, the invention provides methods of treating an inflammatory skin condition in a subject. Representative inflammatory skin conditions treatable by the methods include atopic dermatitis and psoriasis.

In certain embodiments, the method includes reducing the activity of Granzyme K in a subject, thereby treating the skin condition in the subject. In other embodiments, the method includes administering an effective amount of a Granzyme K inhibitor to the subject, thereby treating the inflammatory skin condition in the subject. Suitable Granzyme K inhibitors include small molecules (e.g., organic compounds having a molecular weight less than about 800 g/mole), nucleic acids, peptides, or proteins, such as antibodies. In one embodiment, the Granzyme K inhibitor is an inter-alpha inhibitor protein (IαIp). In another embodiment, the Granzyme K inhibitor is bikunin.

Atopic Dermatitis

The following demonstrates the effectiveness of the methods of the invention for treating atopic dermatitis.

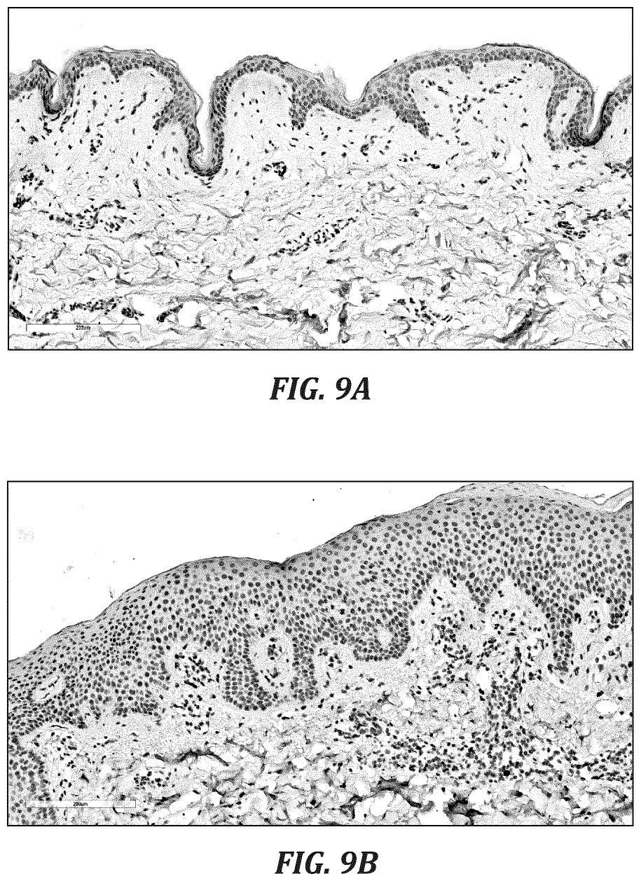

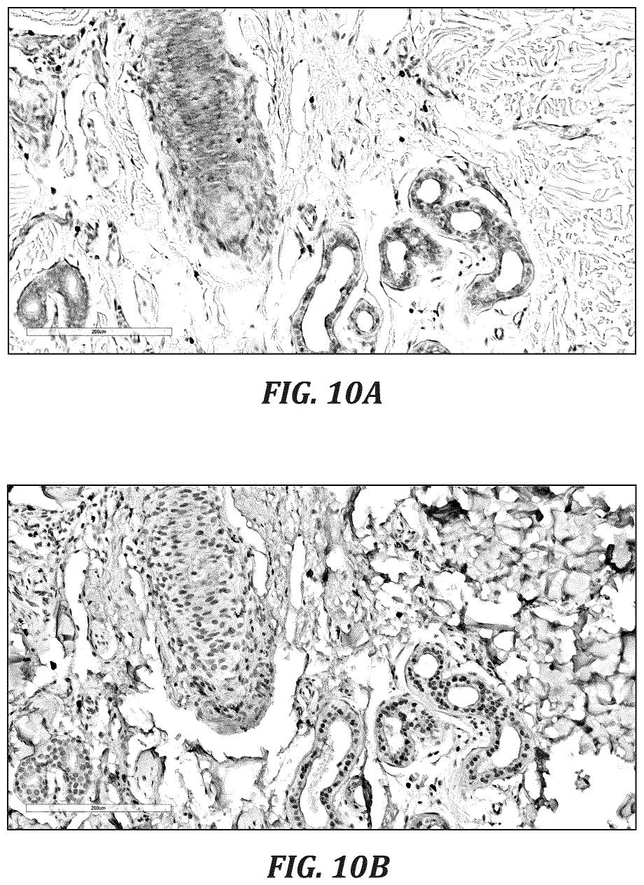

GzmK immunohistochemistry in human lesional atopic dermatitis tissue showing GzmK+ cells elevated in lesional atopic dermatitis tissue (FIG. 9B) compared to healthy skin controls (FIG. 9A). GzmK immunohistochemistry and (FIG. 10B) TBO (mast cell) (FIG. 10A) sequential staining of human atopic dermatitis tissue showing that a majority of mast cells express GzmK, but other cell types also express GzmK.

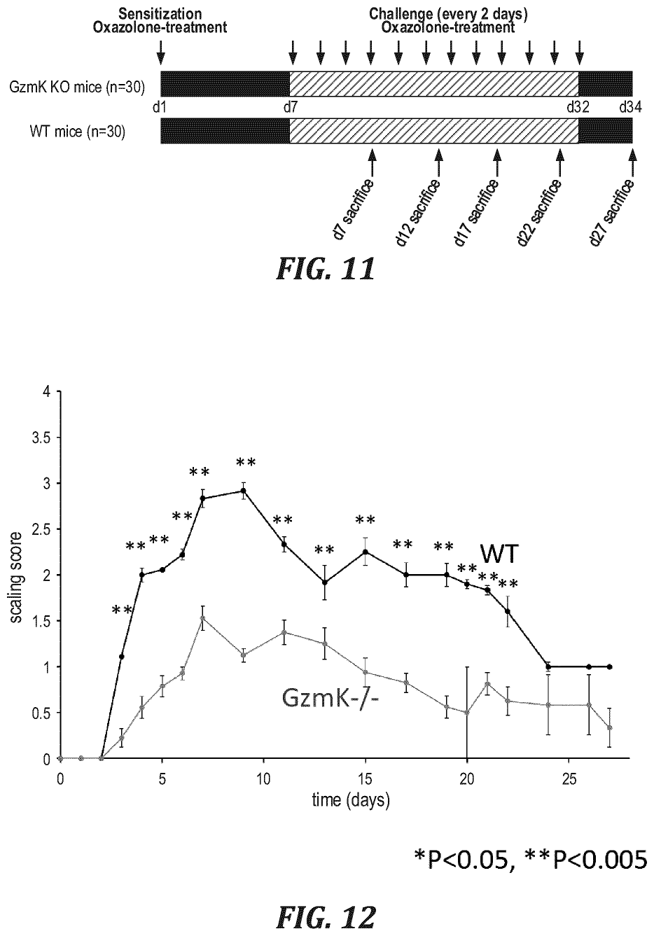

FIG. 11 illustrates the OXA-induced dermatitis mouse model oxazolone exposure schedule described for the experiments described herein. In the hapten-induced dermatitis mouse model mice are sensitized with oxazolone (abdomen and paws). Dermatitis was induced in mice ears with oxazolone seven (7) days later. Exposure of oxazolone was repeated for 27 days (3 times per week). This model variously referred to sub-chronic contact dermatitis or atopic dermatitis (referred to herein as OXA-induced dermatitis). The results from the model are described below.

Scaling was observed to be reduced for GzmK−/− mice compared to WT mice. FIG. 12 illustrates a scaling assessment in the ears of OXA-induced dermatitis mice comparing WT mice and GzmK−/− mice.

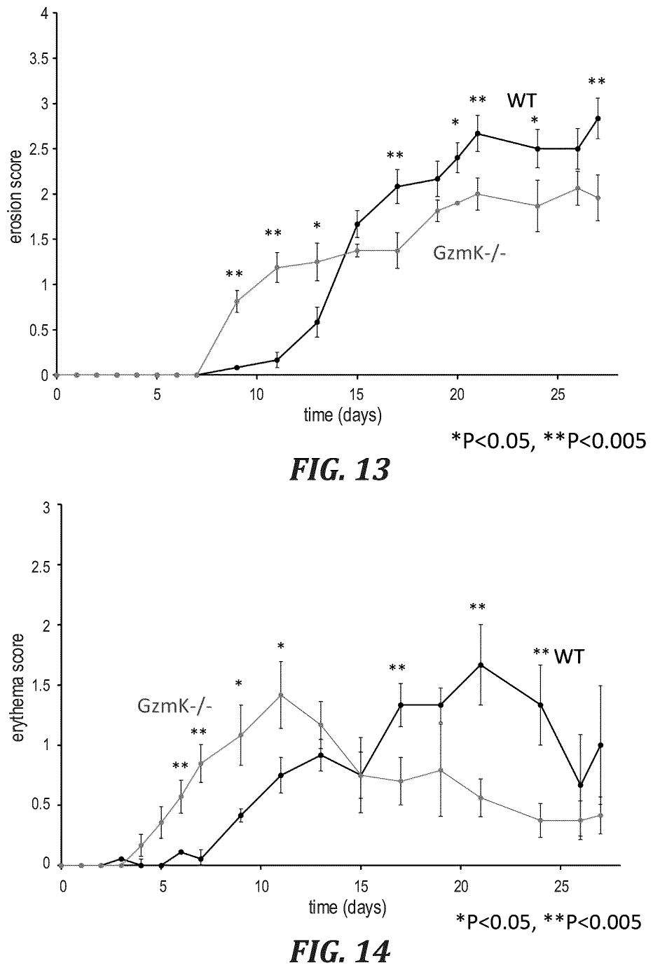

Erosion was observed to be initially worse in GzmK−/− mice, but was significantly reduced from d17 compared to WT controls. FIG. 13 illustrates an erosion assessment in the ears of OXA-induced dermatitis mice comparing WT mice and GzmK−/− mice.

Erythema was observed to be initially worse in GzmK−/− mice, but was reduced from d17 compared to WT controls. FIG. 14 illustrates an erythema assessment in the ears of OXA-induced dermatitis mice comparing WT mice and GzmK−/− mice.

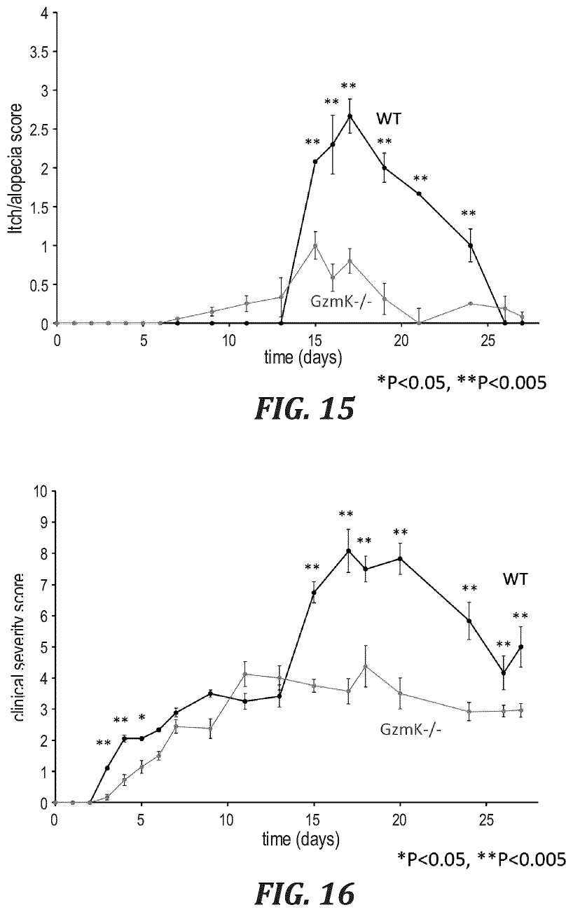

Alopecia was observed to be reduced in the GzmK−/− mice compared to WT controls. FIG. 15 illustrates an alopecia assessment in the ears of OXA-induced dermatitis mice comparing WT mice and GzmK−/− mice.

Severity was observed to be reduced from d15 in the GzmK−/− mice compared to WT controls. FIG. 16 illustrates combined severity score assessment in the ears of OXA-induced dermatitis mice comparing WT mice and GzmK−/− mice.

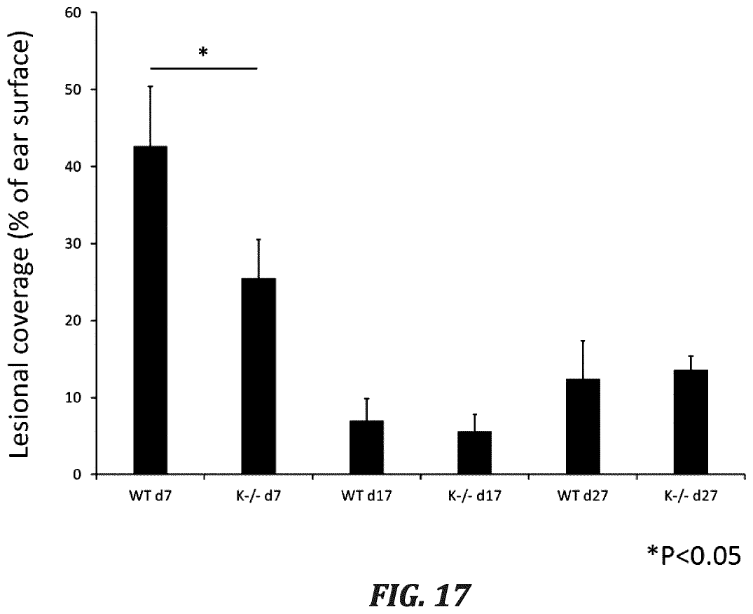

Reduced lesional severity was observed for GzmK−/− mice compared to WT controls. FIG. 17 compares lesional coverage in the ears of OXA-induced dermatitis mice for WT mice and GzmK−/− mice measured from H&E stained ear tissue at d7, d17, and d27.

Psoriasis

The following demonstrates the effectiveness of the methods of the invention for treating psoriasis.

Without being bound to theory, as described herein, GzmK appears to contribute to the onset and progression of psoriasis through the augmentation of inflammation and/or epidermal proliferation.

As described below, GzmK protein level and tissue localization in human psoriasis was characterized, the role of GzmK in psoriasis was assessed using a murine model, and biological pathways and substrates linked to GzmK-mediated pro-inflammatory activity and epidermal proliferation were investigated.

Pre-sectioned human psoriasis biopsies were obtained from Vancouver General Hospital, Vancouver, BC). Biopsies were assessed for GzmK distribution by immunohistochemistry.

GzmK was determined to be elevated in human psoriasis tissue and secreted by immune cells within the dermis. GzmK expression was evaluated in excisional human psoriasis lesions. In healthy skin, GzmK positive cells were minimally dispersed throughout the dermis (see FIG. 28A). In contrast, psoriasis lesional skin exhibited increased number of GzmK positive cells, with the vast majority localized the inflammatory cell infiltrate within the dermis, specifically lymphocytes and cells with dendritic processes (see FIG. 28B). The amount and localization of GzmK positive cells was similar between all three psoriasis samples, despite differences in severity, lesional characteristics, and age of individual.

Using a murine model, the role of GzmK in psoriasis was assessed.

All animal procedures were performed in accordance with the guidelines for animal experimentation approved by the Animal Care Committee of the University of British Columbia. All mice were female with a C57BL/6 background. Wild-type (WT) mice were purchased from Jackson Laboratories at 5 weeks of age. GzmK knockout (KO or GzmK−/−)) mice were bred in-house and age-matched to WT mice.

Using a well-established murine model, 8-11 weeks old GzmK KO and WT mice received a daily topical dose of 62.5 mg of imiquimod (IMQ) cream (5% v/v) directly to the left ear and shaved dorsal skin for a period of 7 (completed) or 14 (completed but awaiting formalin-fixed paraffin-embedded blocks for histologic analysis) consecutive days to promote psoriasis plaque formation. These time points were consistent with the end points used in previous literature and identical to those used in our previous models of cutaneous injury and disease.

High resolution digital pictures of psoriasis plaques of the backs of anesthetized mice were taken from a fixed distance in the presence of a metric ruler and analyzed visually. Every 24 h pre-drug application, pictures were taken of the dorsal region in the presence of a ruler to capture variations in psoriasis lesional severity. Following this, psoriasis severity was quantified using the Psoriasis Severity Index (PSI), which is a quantitative severity assessment based on observable erythema, thickness and squamae. Ear thickness was also measured (using calipers) as a marker of inflammation.

Following euthanasia, the skin area was harvested. The dorsal region was cut in half horizontally. One half was fixed in formalin for 24 h and embedded in paraffin for histological analysis and immunohistochemistry. The other half was flash frozen in liquid nitrogen and stored at −80° C. for analysis of pro-inflammatory cytokines by ELISA and/or Western blot. In addition, 1 mL blood samples were collected by cardiac puncture at euthanasia and centrifuged to obtain plasma for quantification of plasma GzmK levels.

Paraffin-embedded sections were stained with hematoxylin and eosin for evaluation of skin morphology (specifically, epidermal thickness).

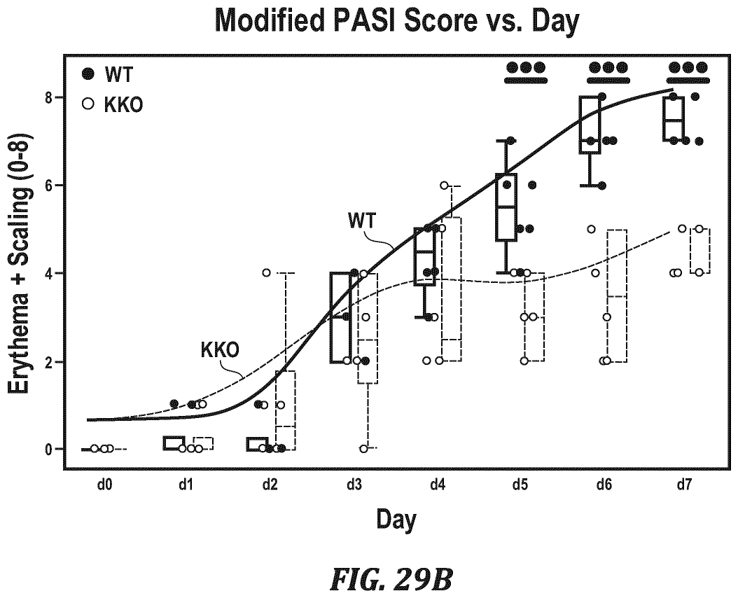

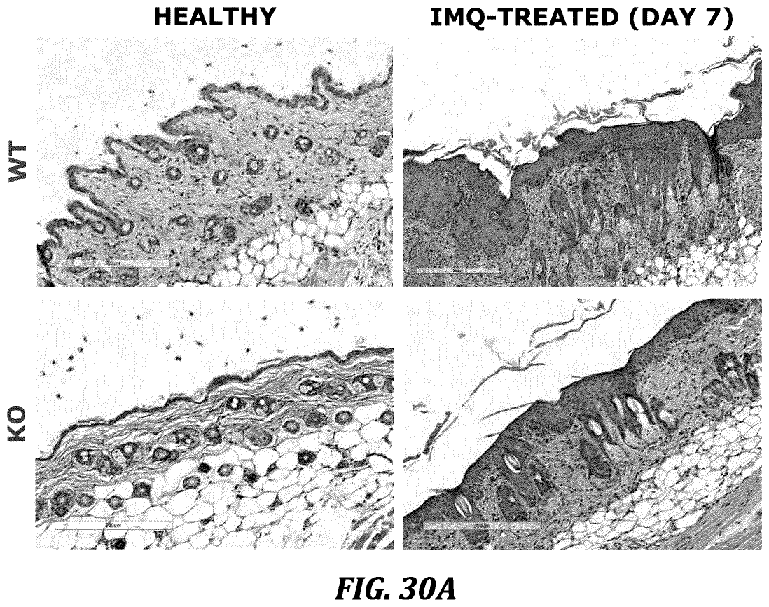

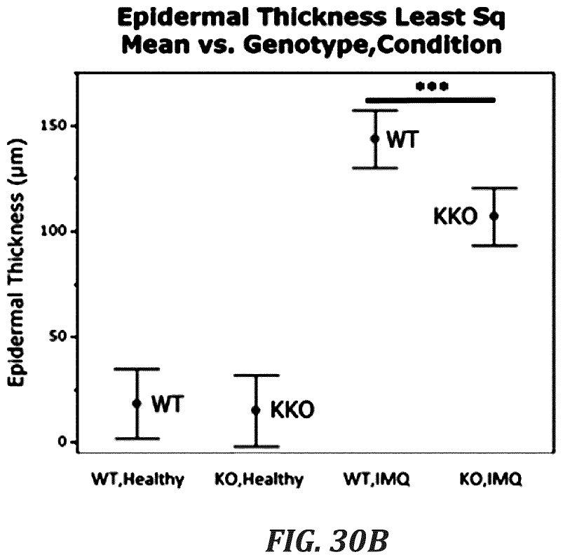

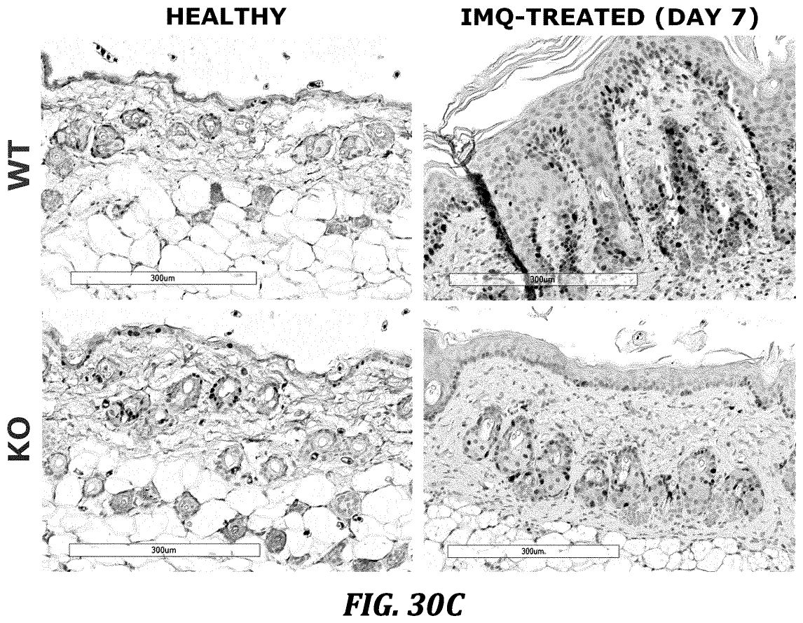

Decreased disease severity was observed for GzmK−/− (KO) mice. GzmK KO and WT mice were subjected to psoriasis lesions on the left ear and dorsal regions. Macroscopically, there was a drastic reduction in erythema, thickness and squamae in GzmK KO mice compared to WT mice at day 7 (see FIGS. 29A and 29B, photographic comparison of drug-induced psoriasis in KO mice and WT mice at day 0 and day 7). Histological assessment supported the macroscopic data, showing a significant reduction in epidermal thickness in GzmK KO mice compared with WT mice at day 7 (see FIGS. 30A and 30B, hematoxylin and eosin stained dorsal tissue of untreated and IMQ-treated WT and untreated and IMQ-treated KO mice at day 7). Additionally, epidermal proliferation marker Ki67 was decreased in IMQ-treated KO vs IMQ-treated WT mice at day 7 (see FIG. 30C, Ki67 immunohistochemistry of dorsal tissue in untreated and IMQ-treated WT and untreated and IMQ-treated KO mice at day 7).

The methods of the invention are demonstrated to be effective in the treatment of inflammatory skin conditions, including atopic dermatitis and psoriasis, where Granzyme K is elevated in the involved tissues.

### Screening Methods

In a further aspect, the invention provides methods for screening a candidate compound for its ability to treat an inflammatory skin condition or to promote wound healing.

In one embodiment, the invention provides a method for screening a candidate compound for its ability to treat an inflammatory skin condition or to promote wound healing, comprising contacting the candidate compound with Granzyme K in vitro, wherein inhibition of Granzyme K activity compared to Granzyme K that has not been contacted with the candidate compound indicates that the candidate compound is a compound that may be useful for the treatment of the inflammatory skin condition or wound. Representative candidate compounds selectively inhibit GzmK and do not substantially inhibit GzmA at the same compound concentration.

### Materials and Methods

Human Samples

Normal human skin and acute burns were obtained from Vancouver General Hospital Burns Clinic with approval from the University of British Columbia Human Research Ethics Committee (H12-00540). Samples were fixed in 10% (v/v) neutral buffered formalin for histology and/or immune-fluorescence.

Cell Culture

THP-1 monocytes were cultured and polarized into M0, M1 and M2a macrophages as described previously (Genin et al., 2015). Primary human skin fibroblasts were from apparently healthy volunteer donated skin biopsies. Fibroblasts and HaCaT cells were maintained in DMEM containing 10% (v/v) FBS and 1% (v/v) penicillin/streptomycin from Sigma-Aldrich (St. Louis, Mo., USA). Cells were cultured in serum-free (HaCaTs) or low serum (2% heat inactivated FBS; fibroblasts and macrophages) medium conditions prior and during each experiment.

Quantitative PCR

RNA was isolated using Trizol Reagent as per manufacturer's directions (Invitrogen, Burlington, ON, Canada). DNase I treatment removed contaminating genomic DNA. cDNA synthesis required random hexamers and M-MuLV reverse transcriptase. cDNA reactions were incubated for 5 minutes at room temperature, 42° C. for 60 minutes, and 65° C. for 20 minutes to inactivate the enzyme. qPCR used PowerUp SYBR master mix on a ViiA in duplicate using primers against:

ICAM1
(SEQ ID NO: 1)
Forward 5′ CTCGAGAGTGGACCCAACTGGAAG 3′
(SEQ ID NO: 2)
Reverse 5′ CAGGCTGGCAGAGGTCTCAG 3′
VCAM1
(SEQ ID NO: 3)
Forward 5′ GAACACTCTTACCTGTGCACAGC 3′
(SEQ ID NO: 4)
Reverse 5′ CTTGACCGTGACCGGCTTCC 3′
MCP1
(SEQ ID NO: 5)
Forward 5′ AACGCCCCACTCACCTGCTG 3′
(SEQ ID NO: 6)
Reverse 5′ CCTTCTTGGGGTCAGCACAG 3′
GAPDH
(SEQ ID NO: 7)
Forward 5′ TGCACCACCAACTGCTTAGC 3′
(SEQ ID NO: 8)
Reverse 5′ GGCATGGACTGTGGTCATGAG 3′

Cycling conditions were: 50° C. 2 minutes 1×, 95° C. 5 minutes 1×, 95° C. 15 seconds, 60° C. 30 seconds 40×. mRNA levels were normalized to GAPDH and compared to WT mice.

Reverse Transcriptase PCR

Total RNA and cDNA synthesis from macrophages was accomplished as described above. Human Granzyme K was amplified using a BioRad T100;

(SEQ ID NO: 9)
Forward 5′ CCTAATAGTTGGGGCTTATATGAC 3′
(SEQ ID NO: 10)
Reverse 5′ GCCTAAAACCACAGTGGGAG 3′)

Thermocycling was as follows: 95° C. 5 minutes 1×, 95° C. 15 seconds, 61° C. 45 seconds 40×, 61° C. 2 minutes. Amplification of GAPDH was used as control. PCR products were separated on a 2% agarose gel and visualized using a LiCOR Odyssey Fc system under the 600 nm channel.

Immunohistochemistry and Immune-Fluorescence

lmmunohistochemistry and immune-fluorescence were performed as previously described (Shen et al., 2012), using the following antibodies: GzmK, human, NovusBio (Oakville, Calif.), 1/300 dilution; GzmK, mouse, NBP2-49387; CD68, human, Abcam (Cambridge, Mass.), ab125212, 1 μg/mL; Collagen I, mouse, Abcam (Cambridge, Mass.), ab34710, 1/200 dilution; Collagen III, mouse, Abcam (Cambridge, Mass.), ab7778, 1/200 dilution; F4/80, mouse, Abcam (Cambridge, Mass.), ab100790, 1/100 dilution; CD3, mouse, Abcam (Cambridge, Mass.), ab5690, 1/100 dilution; and NCR1, mouse, Abcam (Cambridge, Mass.), ab214468, 1/300 dilution.

Morphometric Analysis

Re-epithelialization was measured as (distance of new epithelium from leading edges to wound margins)/(distance of wound bed)×100. Presence of total macrophages, M1 macrophages, T-cells and NK cells were determined by staining intensity in two representative rectangles of 200×160 μm² in the granulation tissue of wound sections (minimum of five wounds on six mice per time point for each group). Data presented as the number of positively stained cells in wounded tissue as a percentage of positively stained cells in WT unwounded skin. There was no difference in cell number between unwounded WT and GzmK−/− skin. Inflammatory infiltrates characterized by high density blue nuclear staining, thus total infiltrate was measured by ratio of blue (nuclear) to red (cytoplasmic) staining (Poo et al., 2014, Wilson et al., 2017).

Electric Cell-Substrate Impedance Sensing

The electrical properties of confluent and wounded fibroblasts and keratinocytes were examined using ECIS (Applied Biophysics, Troy, N.Y., USA), applying the wound assay function as previously described (Turner et al., 2017b). Briefly, cells were seeded into 8W1E PET ECIS Cultureware Arrays (Applied Biophysics, Troy, N.Y., USA) and maintained until confluence. Wells were rinsed twice with PBS, pH 7.2, then incubated for 1 h in FBS-free DMEM, prior to rhGzmK-treatment (10 nM low dose, 25 nM high dose) in FBS-free DMEM. At 1 h, array sensors were wounded at 2500 μA, 48,000 Hz for 20 s. Wound recovery was determined in real time by impedance (36,000 Hz), with recovery defined as the time taken for the signal to plateau.

ELISA

Kit ELISAs were used to evaluate human IL-6 (Human DuoSet ELISA DY206; R&D Systems, Minneapolis, Minn., USA), mouse IL-6 (Rab0309; Sigma-Aldrich, St. Louis, Mo. USA), human IL-1B (ab100562; Abcam, Cambridge, Mass., USA), mouse IL-1B (ab100705; Abcam, Cambridge, Mass., USA) and GzmK (LSBio, Seattle, Wash., USA) in serum-free supernatant from fibroblasts, keratinocytes and macrophage or tissue extracts.

Animal Studies

All procedures performed in accordance with the guidelines approved by the Animal Experimentation Committee, University of British Columbia (A17-0024). GzmK−/− mice (C57Bl/6 background) were generated as described (Joeckel et al., 2017). GzmK−/− mice showed no phenotypic differences to WT mice, including in anatomy, health, fecundity, litter size, and hematopoietic development (Joeckel et al., 2017). C57Bl/6 WT mice obtained from Jackson Laboratories (Bar Harbor, Me., USA) and acclimatized for two weeks prior to commencing experimental procedures. Six female mice (7 to 10 weeks of age) included per treatment group.

Murine Thermal Injury Techniques

Mice were anaesthetized with inhaled isoflurane, and the dorsum shaved and cleaned with 10% (w/v) povidone iodine solution. Thermal injuries were performed by placement of a 6 mm diameter metal rod, heated for 10 minutes in boiling water, on the dorsum for 6 seconds. Digital photographs were captured daily using a ruler aligned next to the wound, allowing direct wound measurements to be made. Wounds were harvested at d3, d6 and d14 and bisected. One half was fixed in 10% (v/v) buffered formalin and processed so that the midpoint of the wound was sectioned and compared between groups. The other half was snap frozen in liquid nitrogen for protein extraction. Additional wounds were harvested at d21 and d41 for skin tensiometry.

Skin Tensiometry

The tensile breaking force of burn wounded skin was evaluated using the Mecmesin Motorised Force Tester (Mecmesin Corporation, Slinfold, UK) similar to reported previously (Kopecki et al., 2013). Briefly, excised skin (1×4 cm; wounded area within the center) was attached to a 200N Spring Action Vice Clamp and pulled apart at 3 cm/minute using the MultiTest 2.5-d Test System Stand. Tensile strength was assessed with an Advanced Force Gauge 100N and recorded in real time using Emperor Lite software. Tensile strength was assessed as the minimum force required to cause skin breakage.

Trypan Blue Exclusion

To evaluate the viability of cultured cells at harvest, a 20 μL aliquot of cell suspension was mixed with an equal volume of 0.1% (v/v) trypan blue and incubated for 5 mins at 20° C. A 20 μL aliquot of the resultant cell suspension was transferred to a haemocytometer and examined at 100× magnification. Greater than 100 cells were counted within five 1 mm² grid squares of the haemocytometer. Non-viable cells were stained blue due to uptake of trypan blue into the cell. Culture viability was evaluated as the percentage of total cells that did not stain blue. Data were not collected from control fibroblast cultures with <90% trypan blue exclusion.

Statistical Analysis

Statistical differences were determined using Student's t-test or 2 way ANOVA. For data not following a normal distribution, the Mann-Whitney U-test was performed. P-values less than 0.05 were considered significant.

### REFERENCES

Bade B, Lohrmann J, Brinke A, Wolbink A M, Wolbink G J, ten Berge I J, et al. Detection of soluble human granzyme K in vitro and in vivo. European journal of immunology 2005; 35(10):2940-8.
Bratke K, Klug A, Julius P, Kuepper M, Lommatzsch M, Sparmann G, et al. Granzyme K: a novel mediator in acute airway inflammation. Thorax 2008; 63(11):1006-11.
Cooper D M, Pechkovsky D V, Hackett T L, Knight D A, Granville D J. Granzyme K activates protease-activated receptor-1. PLoS One 2011; 6(6):e21484.
Farina J A, Jr., Rosique M J, Rosique R G. Curbing inflammation in burn patients. International journal of inflammation 2013; 2013:715645.
Genin M, Clement F, Fattaccioli A, Raes M, Michiels C. M1 and M2 macrophages derived from THP-1 cells differentially modulate the response of cancer cells to etoposide. BMC cancer 2015; 15:577.
Joeckel L T, Allison C C, Pellegrini M, Bird C H, Bird P I. Granzyme K-deficient mice show no evidence of impaired antiviral immunity. Immunology and cell biology 2017.
Joeckel L T, Wallich R, Martin P, Sanchez-Martinez D, Weber F C, Martin S F, et al. Mouse granzyme K has pro-inflammatory potential. Cell Death Differ 2011; 18(7):1112-9.
Joeckel L T, Wallich R, Metkar S S, Froelich C J, Simon M M, Borner C. Interleukin-1R signaling is essential for induction of proapoptotic CD8 T cells, viral clearance, and pathology during lymphocytic choriomeningitis virus infection in mice. Journal of virology 2012; 86(16):8713-9.
Kopecki Z, Ruzehaji N, Turner C, Iwata H, Ludwig R J, Zillikens D, et al. Topically applied flightless I neutralizing antibodies improve healing of blistered skin in a murine model of epidermolysis bullosa acquisita. J Invest Dermatol 2013; 133(4):1008-16.
Ley K, Laudanna C, Cybulsky M I, Nourshargh S. Getting to the site of inflammation: the leukocyte adhesion cascade updated. Nat Rev Immunol 2007; 7(9):678-89.
Lobe C G, Finlay B B, Paranchych W, Paetkau V H, Bleackley R C. Novel serine proteases encoded by two cytotoxic T lymphocyte-specific genes. Science (New Vork, Nev.) 1986; 232(4752):858-61.
Masson D, Nabholz M, Estrade C, Tschopp J. Granules of cytolytic T-lymphocytes contain two serine esterases. The EMBO journal 1986; 5(7):1595-600.
Masson D, Tschopp J. A family of serine esterases in lytic granules of cytolytic T lymphocytes. Cell 1987; 49(5):679-85.
McFarland-Mancini M M, Funk H M, Paluch A M, Zhou M, Giridhar P V, Mercer C A, et al. Differences in wound healing in mice with deficiency of IL-6 versus IL-6 receptor. Journal of immunology (Baltimore, Md.: 1950) 2010; 184(12):7219-28.
Mirza R E, Fang M M, Ennis W J, Koh T J. Blocking interleukin-1beta induces a healing-associated wound macrophage phenotype and improves healing in type 2 diabetes. Diabetes 2013; 62(7):2579-87.
Murray P J. Macrophage Polarization. Annual review of physiology 2017; 79:541-66.
Peck M D. Epidemiology of burns throughout the world. Part I: Distribution and risk factors. Burns 2011; 37(7):1087-100.
Poo V S, Rudd P A, Gardner J, Wilson J A, Larcher T, Colle M A, et al. Multiple immune factors are involved in controlling acute and chronic chikungunya virus infection. PLoS neglected tropical diseases 2014; 8(12):e3354.
Rucevic M, Fast L D, Jay G D, Trespalcios F M, Sucov A, Siryaporn E, et al. Altered levels and molecular forms of granzyme k in plasma from septic patients. Shock (Augusta, Ga.) 2007; 27(5):488-93.
Sharma M, Merkulova Y, Raithatha S, Parkinson L G, Shen Y, Cooper D, et al. Extracellular granzyme K mediates endothelial activation through the cleavage of protease-activated receptor-1. The FEBS journal 2016; 283(9):1734-47.
Shen Y, Guo Y, Mikus P, Sulniute R, Wilczynska M, Ny T, et al. Plasminogen is a key proinflammatory regulator that accelerates the healing of acute and diabetic wounds. Blood 2012; 119(24):5879-87.
Tschopp J, Masson D, Schafer S. Inhibition of the lytic activity of perforin by lipoproteins. J Immunol 1986; 137:1950-3.
Turner C T, Lim D, Granville D J. Granzyme B in skin inflammation and disease. Matrix biology: journal of the International Society for Matrix Biology 2017a.
Turner C T, McInnes S J, Melville E, Cowin A J, Voelcker N H. Delivery of Flightless I Neutralizing Antibody from Porous Silicon Nanoparticles Improves Wound Healing in Diabetic Mice. Advanced healthcare materials 2017b; 6(2).
Voskoboinik I, Whisstock J C, Trapani J A. Perforin and granzymes: function, dysfunction and human pathology. Nat Rev Immunol 2015; 15(6):388-400.
Wensink A C, Wiewel M A, Jongeneel L H, Boes M, van der Poll T, Hack C E, et al. Granzyme M and K release in human experimental endotoxemia. Immunobiology 2016; 221 (7): 773-7.
Wilson J A, Prow N A, Schroder W A, Ellis J J, Cumming H E, Gearing L J, et al. RNA-Seq analysis of chikungunya virus infection and identification of granzyme A as a major promoter of arthritic inflammation. PLoS Pathog 2017; 13{2):e1006155.

While illustrative embodiments have been illustrated and described, it will be appreciated that various changes can be made therein without departing from the spirit and scope of the invention.

1. A method of treating an inflammatory skin condition in a subject, comprising reducing the activity of Granzyme K in a subject, thereby treating the inflammatory skin condition.

2. The method of claim 1, wherein the inflammatory skin condition is psoriasis or atopic dermatitis.

3. A method of treating a wound in a subject, comprising reducing the activity of Granzyme K in a subject, thereby treating the wound.

4. The method of claim 3, wherein the wound is a burn wound, chronic wound, acute wound, pressure injury, or ischemic injury.

5. The method of claim 4, wherein the pressure injury is ischemia reperfusion injury.

6. The method of claim 1, wherein reducing the activity of Granzyme K comprises administering an effective amount of a Granzyme K inhibitor to the subject.

7. The method of claim 6, wherein the Granzyme K inhibitor is a small molecule, a nucleic acid molecule, a peptide, or an antibody.

8. The method of claim 6, wherein the Granzyme K inhibitor is an inter-alpha inhibitor protein (IαIp).

9. The method of claim 6, wherein the Granzyme K inhibitor is bikunin.

10. A method of treating an inflammatory skin condition in a subject, comprising administering an effective amount of a Granzyme K inhibitor to the subject, thereby treating the inflammatory skin condition.

11. The method of claim 10, wherein the inflammatory skin condition is psoriasis or atopic dermatitis.

12. A method of treating a wound in a subject, comprising administering an effective amount of a Granzyme K inhibitor to the subject, thereby treating the wound.

13. The method of claim 12, wherein the wound is a burn wound, acute wound, chronic wound, pressure injury, or ischemic injury.

14. The method of claim 13, wherein the pressure injury is ischemia reperfusion injury.

15. The method of claim 10, wherein the Granzyme K inhibitor is a small molecule, a nucleic acid molecule, a peptide, or an antibody.

16. The method of claim 10, wherein the Granzyme K inhibitor is an inter-alpha inhibitor protein (IαIp).

17. The method of claim 10, wherein the Granzyme K inhibitor is bikunin.

18. A method of promoting wound healing in a subject, comprising inhibiting cleavage of syndecan-1 in keratinocytes by reducing the activity of Granzyme K in the subject.

19. A method of promoting wound healing in a subject, comprising reducing pro-inflammatory cytokine response in keratinocytes, fibroblasts, macrophages, and/or endothelial cells by reducing the activity of Granzyme K in the subject.

20. A method for promoting wound re-epithelization, comprising reducing the activity of Granzyme K in keratinocytes proximate to the wound.

21. A method of promoting wound healing in a subject, comprising inhibiting cleavage of syndecan-1 by administering an effective amount of Granzyme K inhibitor to the subject.

22. A method of promoting wound healing in a subject, comprising reducing pro-inflammatory cytokine response in keratinocytes, fibroblasts, macrophages, and/or endothelial cells by administering an effective amount of Granzyme K inhibitor to the subject.

23. A method for promoting wound re-epithelization in a subject, comprising inhibiting cleavage of syndecan-1 in a keratinocyte by administering an effective amount of Granzyme K inhibitor to the subject.

24. A method for promoting wound re-epithelization in a subject, comprising administering an effective amount of Granzyme K inhibitor to the subject.

25. The method of claim 18, wherein the wound is a burn wound, chronic wound, acute wound, pressure injury, or ischemic injury.

26. The method of claim 21, wherein the inhibitor is administered topically or systemically.

27. The method of claim 21, wherein the Granzyme K inhibitor is a small molecule, a nucleic acid molecule, a peptide, or an antibody.

28. The method of claim 21, wherein the Granzyme K inhibitor is an inter-alpha inhibitor protein (IαIp).

29. The method of claim 21, wherein the Granzyme K inhibitor is bikunin.

30. A method of stimulating re-epithelialization, comprising inhibiting syndecan-1 cleavage in the keratinocyte by reducing the activity of GzmK in the wounded or damaged tissue area.

31. A method of converting a pro-inflammatory phenotype to a pro-healing wound repair phenotype, comprising reducing pro-inflammatory cytokine responses in keratinocytes, fibroblasts, macrophages, and/or endothelial cells by reducing the activity of GzmK in the wounded or damaged tissue area.

32. A method for screening a candidate compound for its ability to treat an inflammatory skin condition or to promote wound healing, comprising contacting the candidate compound with Granzyme K in vitro, wherein inhibition of Granzyme K activity compared to Granzyme K that has not been contacted with the candidate compound indicates that the candidate compound is a compound that may be useful for the treatment of the inflammatory skin condition or wound.

33. The method of claim 32, wherein the candidate compound selectively inhibits GzmK and does not substantially inhibit GzmA at the same compound concentration.
